(19) United States
(12) Patent Application Publication
(10) Pub. No.: US 2025/0255873 A1
(43) Pub. Date: Aug. 14, 2025

(54) RESTORING FUNCTION OF SPECIFIC MUTATIONS AFFECTING THE ACTIN DYNAMIC BY ADMINISTRATION OF SAPROPTERIN

(71) Applicant: QRGenetics Ltd., Tel Aviv (IL)

(72) Inventors: Shane Wald-Altman, Ramat-Hasharon (IL); Noa Bielopolski, Tel Aviv (IL)

(21) Appl. No.: 19/182,228

(22) Filed: Apr. 17, 2025

Related U.S. Application Data
(63) Continuation of application No. PCT/IB2023/060542, filed on Oct. 18, 2023.
(60) Provisional application No. 63/416,973, filed on Oct. 18, 2022.

(51) Int. Cl. A61K 31/519; A61K 45/06; A61P 9/00; C12Q 1/6883

(52) U.S. Cl. CPC A61K 31/519; A61K 45/06; A61P 9/00; C12Q 1/6883; C12Q 2600/156

(57) ABSTRACT

The present invention provides methods of treatment, diagnostics, kits, and formulations for treating a subject having one or more genetic variations in an Actin alpha 2 (ACTA2) gene involving administering a therapeutically effective amount of a pterin of Formula I such as sapropterin to a subject, contingent upon a presence of one or more genetic variations in Actin alpha 2 (ACTA2) gene.

### RELATED APPLICATIONS

The present patent application is a Continuation of International Application No. PCT/IB2023/060542 filed on 18 Oct. 2023 which is incorporated herein by reference in its entirety. PCT/IB2023/060542 claims the benefit of U.S. Application Ser. No. 63/416,973, which was filed on Oct. 18, 2022, and which is incorporated herein by reference in its entirety.

### SEQUENCE LISTING

The application contains a Sequence Listing which has been submitted electronically in XML format and is hereby incorporated by reference in its entirety. Said XML copy, created on filed Dec. 2, 2024, is named QRG0019US01 Seq listing and is 2 kilobytes in size.

### FIELD OF THE INVENTION

The present invention relates to predictive methods, personalized therapies, kits, and the use of pterin derivatives (e.g., biopterin) for the treatment of vascular diseases associated with Actin Alpha 2 (ACTA2) such as premature onset coronary artery disease, premature ischemic strokes, Moyamoya disease and aortic aneurysms including Familial Ascending Thoracic Aortic Aneurysms.

### BACKGROUND TO THE INVENTION

Aortic aneurysm disease remains a silent killer, with the majority of patients dying from complications such as rupture or dissection of an aneurysm. An aortic aneurysm is defined as a permanent localized dilatation of the aorta that is more than 50% of the predicted, resulting in weakness in the wall while causing minimal symptoms until ruptured. When a rupture occurs, massive internal bleeding results and, unless treated immediately, shock and death can occur. It is usually developed in weak locations of the aorta and is classified into subgroups, namely thoracic aortic aneurysm (TAA) or abdominal aortic aneurysm (AAA). Worldwide it is estimated that aortic aneurysm-related mortality is estimated at 150,000-200,000 deaths per year. Unfortunately to date, there is no effective medication to prevent or reverse the progression of the disease (Zhuo et al., 2019).

The actin microfilament network is important in maintaining cell shape and function in eukaryotic cells. It has a variety of roles in cellular processes such as cellular signalling, cell adhesion, muscle contraction, motility, intracellular trafficking and cytokinesis. Actin is a highly conserved protein and is ubiquitously expressed in all eukaryotic cells. In vertebrates, three main groups of actin isoforms have been identified, namely alpha, beta and gamma. The alpha actins are found in muscle tissues and are a major constituent of the contractile apparatus. The beta and gamma actins coexist in most cell types as components of the cytoskeleton and as mediators of internal cell motility. The gene encoding alpha smooth muscle actin, Actin Alpha 2 (herein referred to as ACTA2) gene (chromosome location 10q23.31) predisposes patients to a group of diffuse vascular diseases, including premature onset coronary artery disease, premature ischemic strokes, aortic aneurysms, and Moyamoya disease (Guo D C et. al., 2007). ACTA2 is mutated in some cases of familial thoracic aortic aneurysms (FTAA) which have an insidious course and account for 10-15% of all FTAA. (Regalado E. S., et. al., 2015). In theory, the inability of mutant forms of ACTA2 to interface with the β myosin heavy chain (encoded by myh11) due to loss of structural integrity, leads to a loss of contractile force in aortic vascular smooth muscle cells. Studies predict that cells expressing a mutation will have a larger pool of monomeric actin. These dysfunctions could trigger aberrant mechano-sensing pathways that culminate in compromised aortic muscle tissue (Humphrey J D et. al., 2015). Increasing the monomer/polymer ratio of actin may also have an impact on cellular phenotype, which could have implications for occlusive vascular disease. Indeed, individuals with ACTA2 mutations show substantially fewer actin filaments than controls, suggesting that these mutations may interfere with actin assembly (Guo, D. C., 2007).

Various pterin derivatives have been researched with application in multiple areas ranging from neurological, respiratory, and vascular diseases. Fosdenopterin (Nulibry®) was approved in 2021 for the treatment of a rare disease by the name of molybdenum cofactor deficiency type A. Tetrahydrobiopterin (e.g., Sapropterin), was first approved in 2007 as Kuvan® for the treatment of hyperphenylalaninemia caused by tetrahydrobiopterin responsive phenylketonuria. Tetrahydrobiopterin is a pterin derivative belonging to a class of biopterin, which are naturally occurring essential cofactors of critical intracellular enzymes such as phenylalanine hydroxylase (PAH), tyrosine hydroxylase (TH), tryptophan hydroxylase, nitric oxide synthases (NOS), and alkylglycerol monooxygenase (AGMO). U.S. Pat. No. 4,778,794, discloses that tetrahydrobiopterin can ameliorate disorders of infantile autism. U.S. Pat. No. 8,124,653, discloses that tetrahydropterin can be used to treat a patient with attention deficit hyperactivity disorder (ADHD). U.S. Pub. No. 20070049599 discloses that tetrahydrobiopterin can prevent and/or treat a respiratory disease. WO2006063215 discloses that tetrahydrobiopterin can treat an infant having below normal arterial oxygen pressure (PaO2). WO9532203 discloses that inhibitory pteridine derivatives are capable of prevention and treatment of pathological blood pressure decrease, colitis ulcerosa, myocardial infarction, transplant rejection, Morbus Alzheimer, epilepsy and migraine. WO2007050585 discloses that tetrahydrobiopterin can be used to treat cardiac disease or disorders in subjects. Despite multiple potential applications for this group of molecules, to date, only a couple of pterin derivatives have been approved by the FDA.

Thus, there remains a need for novel, accurate and non-invasive treatment for patients having, or at risk for developing vascular diseases and especially those patients with associated ACTA2 genetic variation including but not limited to premature onset coronary artery disease, premature ischemic strokes and Moyamoya disease and especially aortic aneurysms. More specifically, it would be beneficial to remedy the deficient activity of actin and return functional properties of actin in vascular diseases associated with actin mutations including, for example, a potential or developing aortic aneurysm. The kits, compositions and methods described herein can be used to generate a corrected actin protein functionality despite the presence of the specific mutations.

### SUMMARY OF THE INVENTION

Provided are methods of treatment, diagnostics, kits, combinations and methods of manufacture which remedy the long-felt need for improved treatment of a subset of vascular diseases. The subset of vascular diseases of the present invention include those associated with a subset of ACTA2 mutations, such as premature onset coronary artery disease, premature ischemic strokes, Moyamoya disease and aortic aneurysms. The kits, therapeutic agents and methods provided are based, at least in part, on the discovery that a set of pterin derivatives, having the general Formula I, for example Formula (Ia), (Ib), (Ic) or specifically Sapropterin, are capable of improving or compensating for dysfunctional activity of ActinA2, in a subset of the vascular diseases associated with specific ACTA2 mutations, such as premature onset coronary artery disease, premature ischemic strokes, Moyamoya disease, MSMDS and aortic aneurysms (e.g., familial thoracic aortic aneurysms patients).

In a first aspect, there is provided a method for treating a subject having one or more genetic variations in an Actin alpha 2 (ACTA2) gene, comprising: administering a therapeutically effective amount of pterin Formula I (e.g., Sapropterin) to a subject, contingent upon the presence of one or more genetic variations. In some embodiments, the one or more genetic variations in the Actin alpha 2 (ACTA2) gene is a minor allele of a single nucleotide polymorphism (SNP) from selected the list consisting of: rs112602953, rs112901682, rs121434526, rs 121434527, rs121434528, rs387906592, rs397515325, rs397516683, rs397516685, rs727502878, rs746972765, rs772862676, rs794728019, rs794728021, rs794728025, rs794728029, rs869025352, rs886038852, rs886038978, rs886039303, rs1057521105, rs1060500134, rs1064793016, rs1254836237, rs1554841843 and rs150547139.

In various embodiments, the genetic variation is any genetic variation in the actin alpha 2 protein which does not affect the predicted 3-dimensional orientation of HIS H: 75 or ILE A: 78 and ARG R: 179, LYS K: 193 or THR T: 196 in the resulting actin alpha 2 protein.

In another aspect of the present invention, a method of treatment is provided comprising administering to a patient having an aortic aneurysm, Moyamoya disease 5 or Multisystemic smooth muscle dysfunction syndrome (MSMDS) a pterin Formula I. In some embodiments, the pterin is Formula Ia. In some embodiments, the pterin is Formula Ib. In some embodiments, the pterin is Formula Ic. In some embodiments, the pterin is Formula Id. In some embodiments, the pterin is sapropterin.

In another aspect, there is provided a method for treating a subject diagnosed with or in a risk category for developing a vascular disease, the method comprising: obtaining a biological sample from the subject, detecting in the biological sample a presence of one or more genetic variations being substitutions in an actin alpha (ACTA2) locus; and administering a therapeutically effective amount of pterin Formula I or pharmaceutically acceptable salt or polymorph thereof to the subject when the one or more genetic variations are present.

In another aspect, there is provided a method of treating a subject diagnosed with or in a risk category for developing an ascending thoracic aortic aneurysm, the method comprising: obtaining a biological sample from the subject
detecting in the biological sample the presence of one or more substitutions in the actin alpha (ACTA2) locus selected from: a substitution that results in an amino acid change consisting of a substitution of arginine (R) for proline (P) at position 118 (R118P) or a proline (P) allele of a single nucleotide polymorphism SNP of rs112602953; a substitution that results in an amino acid change consisting of a substitution of arginine for glutamine at position 118 (R118Q) or a glutamine (Q) allele of a single nucleotide polymorphism SNP of rs112602953; a substitution that results in an amino acid change consisting of a substitution of arginine for cysteine at position 149 (R149C) or a cysteine (C) allele of a single nucleotide polymorphism SNP of rs 121434526; a substitution that results in an amino acid change consisting of a substitution of arginine for histidine at position 179 (R179H) or a histidine (H) allele of a single nucleotide polymorphism SNP of rs387906592; a substitution that results in an amino acid change consisting of a substitution of arginine for cysteine at position 258 (R258C) or a cysteine (C) allele of a single nucleotide polymorphism SNP of rs121434528; and administering a therapeutically effective amount of pterin Formula I or pharmaceutically acceptable salt or polymorph thereof to the subject.

In various embodiments, the genetic variation in the Actin alpha 2 (ACTA2) is a minor allele of a single nucleotide polymorphism (SNP) selected from the list consisting of: rs 112602953, rs112901682, rs121434526, rs121434527, rs121434528, rs387906592, rs397515325, rs397516683, rs397516685, rs727502878, rs746972765, rs772862676, rs794728019, rs794728021, rs794728025, rs794728029, rs869025352, rs886038852, rs886038978, rs886039303, rs1057521105, rs1060500134, rs1064793016, rs1254836237, rs 1554841843 and rs150547139.

In various embodiments, the genetic variation is any genetic variation in the actin alpha 2 protein which does not affect the predicted 3-dimensional orientation of HIS H: 75 or ILE A: 78 and ARG R: 179, LYS K: 193 or THR T: 196 in the resulting actin alpha 2 protein.

In various embodiments, a genetic variation may be the presence of one or more substitutions in the actin alpha (ACTA2) locus selected from: a substitution that results in an amino acid change consisting of a substitution of arginine (R) for histidine (H) at position 258 (R258H) or a histidine (H) allele of a single nucleotide polymorphism (SNP) of rs121434527; a substitution that results in an amino acid change consisting of a substitution of methionine (M) for valine (V) at position 49 (M49V) or a valine (V) allele of a single nucleotide polymorphism (SNP) of rs397515325; a substitution that results in an amino acid change consisting of a substitution of alanine (A) for valine (V) at position 140 (A140V) or a valine (V) allele of a single nucleotide polymorphism (SNP) of rs397516683; a substitution that results in an amino acid change consisting of a substitution of arginine (R) for glutamine (Q) at position 212 (R212Q) or a glutamine (Q) allele of a single nucleotide polymorphism (SNP) of rs397516685; a substitution that results in an amino acid change consisting of a substitution of lysine (K) for asparagine (N) at position 240 (K240N) or a asparagine (N) allele of a single nucleotide polymorphism (SNP) of rs727502878; a substitution that results in an amino acid change consisting of a substitution of arginine (R) for histidine (H) at position 198 (R198H) or a histidine (H) allele of a single nucleotide polymorphism (SNP) of rs746972765; a substitution that results in an amino acid change consisting of a substitution of arginine (R) for cysteine (C) at position 198 (R198C) or a cysteine (C) allele of a single nucleotide polymorphism (SNP) of rs772862676; a substitution that results in an amino acid change consisting of a substitution of methionine (M) for valine (V) at position 1 (M1V) or a valine (V) allele of a single nucleotide polymorphism (SNP) of rs794728019; a substitution that results in an amino acid change consisting of a substitution of arginine (R) for histidine (H) at position 39 (R39H) or a histidine (H) allele of a single nucleotide polymorphism (SNP) of rs794728021; a substitution that results in an amino acid change consisting of a substitution of arginine (R) for histidine (H) at position 149 (R149H) or a histidine (H) allele of a single nucleotide polymorphism (SNP) of rs794728025; a substitution that results in an amino acid change consisting of a substitution of arginine (R) for leucine (L) at position 149 (R149L) or a leucine (L) allele of a single nucleotide polymorphism (SNP) of rs794728025; a substitution that results in an amino acid change consisting of a substitution of glycine (G) for glutamic acid (E) at position 270 (G270E) or a glutamic acid (E) allele of a single nucleotide polymorphism (SNP) of rs794728029; a substitution that results in an amino acid change consisting of a substitution of methionine (M) for lysine (K) or threonine (T) at position 49 (M49K or M49T) or alternatively, a lysine (K) or threonine (T) allele of a single nucleotide polymorphism (SNP) of rs869025352; a substitution that results in an amino acid change consisting of a substitution of arginine (R) for cysteine (C) or serine(S) at position 256 (R256C or R256S) or alternatively, a cysteine (C) or serine(S) allele of a single nucleotide polymorphism (SNP) of rs886038852; a substitution that results in an amino acid change consisting of a substitution of arginine (R) for cysteine (C) or serine(S) at position 179 (R179C or R179S) or alternatively, a cysteine (C) or serine(S) allele of a single nucleotide polymorphism (SNP) of rs886039303; a substitution that results in an amino acid change consisting of a substitution of arginine (R) for glutamine (Q) at position 185 (R185Q) or a glutamine (Q) allele of a single nucleotide polymorphism (SNP) of rs1057521105; a substitution that results in an amino acid change consisting of a substitution of proline (P) for leucine (L) at position 72 (P72L) or a leucine (L) allele of a single nucleotide polymorphism (SNP) of rs1060500134; a substitution that results in an amino acid change consisting of a substitution of glycine (G) for serine(S) at position 76 (G76S) or a serine(S) allele of a single nucleotide polymorphism (SNP) of rs1064793016; a substitution that results in an amino acid change consisting of a substitution of aspartic acid (D) for glutamic acid (E) at position 82 (D82E) or alternatively, a glutamic acid (E) allele of a single nucleotide polymorphism (SNP) of rs1254836237; a substitution that results in an amino acid change consisting of a substitution of methionine (M) for arginine (R) at position 46 (M46R) or alternatively, an arginine (R) allele of a single nucleotide polymorphism (SNP) of rs1554841843; a substitution that results in an amino acid change consisting of a substitution of aspartic acid (D) for glutamic acid (E) at position 58 (D58E) or a glutamic acid (E) allele of a single nucleotide polymorphism (SNP) of rs 150547139; a substitution that results in an amino acid change consisting of a substitution of arginine (R) for proline (P) at position 118 (R118P) or a proline (P) allele of a single nucleotide polymorphism SNP of rs112602953; a substitution that results in an amino acid change consisting of a substitution of arginine for glutamine at position 118 (R118Q) or a glutamine (Q) allele of a single nucleotide polymorphism SNP of rs112602953; a substitution that results in an amino acid change consisting of a substitution of arginine for cysteine at position 149 (R149C) or a cysteine (C) allele of a single nucleotide polymorphism SNP of rs121434526; a substitution that results in an amino acid change consisting of a substitution of arginine for histidine at position 179 (R179H) or a histidine (H) allele of a single nucleotide polymorphism SNP of rs387906592; a substitution that results in an amino acid change consisting of a substitution of arginine for cysteine at position 258 (R258C) or a cysteine (C) allele of a single nucleotide polymorphism SNP of rs121434528.

In various embodiments, the genetic variation may be one or more substitutions in an actin alpha (ACTA2) locus selected from: a substitution that results in an amino acid change consisting of a substitution of arginine (R) for proline (P) at position 118 (R118P) or a proline (P) allele of a single nucleotide polymorphism SNP of rs112602953; a substitution that results in an amino acid change consisting of a substitution of arginine for glutamine at position 118 (R118Q) or a glutamine (Q) allele of a single nucleotide polymorphism SNP of rs112602953; a substitution that results in an amino acid change consisting of a substitution of arginine for cysteine at position 149 (R149C) or a cysteine (C) allele of a single nucleotide polymorphism SNP of rs121434526; a substitution that results in an amino acid change consisting of a substitution of arginine for histidine at position 179 (R179H) or a histidine (H) allele of a single nucleotide polymorphism SNP of rs387906592; a substitution that results in an amino acid change consisting of a substitution of arginine for cysteine at position 258 (R258C) or a cysteine (C) allele of a single nucleotide polymorphism SNP of rs121434528.

In various embodiments, the effective amount of pterin is an amount sufficient to cause in the subject, any one or more of the following, subsequent to treatment: an increase in functional F-actin (e.g., such as a quantitative improvement in subject's functional activity of actin or an increased functional F-actin formation); an increase in a ratio between F (fibrous actin polymerized in the form of a double helix) to G (globular monomeric form) in the subject; degradation or decrease G-actin formation; in the subject; an increase in contractile force in aortic vascular smooth muscle cells; and improvement in edema.

In various embodiments, the subject is diagnosed with or in a risk category for developing a vascular disease selected from a list consisting of: premature onset coronary artery disease, premature ischemic strokes, Moyamoya disease, MSMDS and aortic aneurysm. In various embodiments, the subject is diagnosed with or in a risk category for developing a vascular disease selected from a list consisting of: premature onset coronary artery disease, premature ischemic strokes, Moyamoya disease and aortic aneurysm. For example, the vascular disease may be an aneurysm or more specifically, an aortic aneurysm such as a descending aortic aneurysm, an ascending aortic aneurysm, and/or an abdominal aortic aneurysm. The ascending aortic aneurysm may be an ascending thoracic aortic aneurysm. The vascular disease may be Multisystemic smooth muscle dysfunction syndrome (MSMDS). The vascular disease may be premature onset coronary artery disease. The vascular disease may be premature ischemic strokes. The vascular disease may be Moyamoya disease.

In various embodiments, the subject is a fetus, a child, or an adult. For example, the child may be less than one year old. In some embodiments, the subject is a fetus and administering is to a pregnant adult for exposure to a developing fetus.

The pterin derivative, as used herein in accordance with aspects and embodiments of the present invention has the following general formula in free base or pharmaceutically acceptable salt form or/and polymorphs thereof:

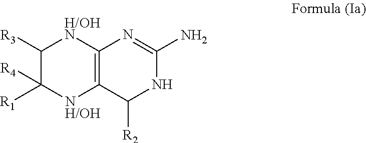

wherein
NH/OH represents an amine or hydroxylamine (i.e., nitrogen atom, further bound to a hydrogen or hydroxyl group),
R1 represents a hydrogen, alkyl (e.g., methy), hydroxyalkyl, hydroxyl, carbonyl, phosphate, ether or ketone group,
R2 represents an oxygen or amine group,
R3 represents an oxygen, hydrogen, methyl, or hydroxyl group and
R4 represents an oxygen, hydrogen, methyl, or hydroxyl group.

The pterin derivative, as used herein in accordance with aspects and embodiments of the present invention, may have the following formula (Ib) in free base or pharmaceutically acceptable salt form or/and polymorphs thereof:

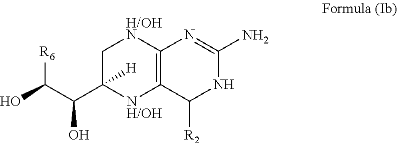

wherein
NH/OH represents an amine or hydroxylamine (i.e., nitrogen atom, further bound to a hydrogen or hydroxyl group),
O/OH represents a hydroxyl group, or an oxygen atom on a double bond,
R6 represents an alkyl (e.g., methyl), hydroxyalkyl, hydroxyl, carbonyl, phosphate, ketone, or ether, and
R2 represents an oxygen or amine group.

In Formula Ia and Ib (herein Formula I), R1 or R6 may be selected from the list consisting of the following: CH₂OH, CH₂CH₂OH, CH₃, CH₂CH₃, HCOHCH₂OH, CHOHCH₃, CHOHCH₂OH, COCH₃, OH, O, CH₂CH₂OH, CHOHCH₂P₃O₁₀H₄. In some embodiments, R2 is selected from the list consisting of the following: NH2, O or OH. Numerous combinations of R1 and R2 can also be envisioned.

The pterin derivative, as used herein in accordance with aspects and embodiments of the present invention, may have the following formula (Ic) in free base or pharmaceutically acceptable salt form or/and polymorphs thereof:

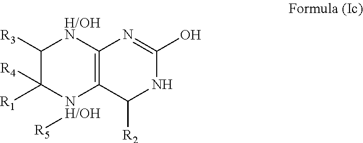

wherein
NH/OH represents an amine or hydroxylamine (i.e., nitrogen atom, further bound to a hydrogen or hydroxyl group),
R1 represents a hydrogen, alkyl (e.g., methy), hydroxyalkyl, hydroxyl, carbonyl, phosphate, ether or ketone group,
R2 represents an oxygen or hydroxyl group,
R3 represents an oxygen, hydrogen, methyl or hydroxyl group,
R4 represents an oxygen, hydrogen, methyl or hydroxyl group, and
R5 represents a hydroxyl or hydroxyl alcohol.

In some embodiments of the present invention, the pterin Formula I is (6R)-6-lactoyl-5,6,7,8-tetrahydropterin or a pharmaceutically acceptable salt or/and polymorph thereof. In some embodiments of the present invention, the pterin Formula I is (6R)-5,6,7,8-tetrahydrobiopterin or a pharmaceutically acceptable salt or/and polymorph thereof. In some embodiments of the present invention, the pterin Formula I is 5,6,7,8-tetrahydrobiopterin or a pharmaceutically acceptable salt or/and polymorph thereof. In some embodiments of the present invention, the pterin Formula I is sepiapterin or a pharmaceutically acceptable salt or/and polymorph thereof. In some embodiments of the present invention, the pterin Formula I is 6-Methyltetrahydropterin or a pharmaceutically acceptable salt or/and polymorph thereof. In some embodiments of the present invention, the pterin Formula I is Hydroxysepiapterin or a pharmaceutically acceptable salt or/and polymorph thereof. In some embodiments of the present invention, the pterin Formula I is 7,8-dihydroneopterin or a pharmaceutically acceptable salt or/and polymorph thereof. In some embodiments of the present invention, the pterin Formula I is 6-lactoyl-5,6,7,8-tetrahydropterin or a pharmaceutically acceptable salt or/and polymorph thereof. In some embodiments of the present invention, the pterin Formula I is Tetrahydroneopterin or a pharmaceutically acceptable salt or/and polymorph thereof. In some embodiments of the present invention, the pterin Formula I is leucopterin (keto form) or a pharmaceutically acceptable salt or/and polymorph thereof. In some embodiments of the present invention, the pterin Formula I is 1-hydroxy-2-Oxopropyl tetrahydropterin or a pharmaceutically acceptable salt or/and polymorph thereof. In some embodiments of the present invention, the pterin Formula I is 6,7-Dimethyltetrahydropterin or a pharmaceutically acceptable salt or/and polymorph thereof. In some embodiments of the present invention, the pterin Formula I is 2-amino-5,6,7,8-tetrahydroxy-6-(1,2,3-trihydroxypropyl)-3,7-dihydropteridin-4-one or a pharmaceutically acceptable salt or/and polymorph thereof. In some embodiments of the present invention, the pterin Formula I is sapropterin dihydrochloride or a pharmaceutically acceptable salt or/and polymorph thereof. In some embodiments of the present invention, the pterin Formula I is L-erythro-7,8-dihydrobiopterin or a pharmaceutically acceptable salt or/and polymorph thereof. In some embodiments of the present invention, the pterin Formula I is 2-amino-6-(hydroxymethyl)-7,8-dihydropteridin-4-one or a pharmaceutically acceptable salt or/and polymorph thereof. In some embodiments of the present invention, the pterin Formula I is tetrahydrodictyopterin or a pharmaceutically acceptable salt or/and polymorph thereof. In some embodiments of the present invention, the pterin Formula I is sapropterin or a pharmaceutically acceptable salt or/and polymorph thereof. In some embodiments of the present invention, the pterin Formula I is 5,6,7,8-tetrahydropterin-6-carboxylate or a pharmaceutically acceptable salt or/and polymorph thereof. In some embodiments of the present invention, the pterin Formula I is 5,6,7,8-tetrahydropterin-6-carboxylic acid or a pharmaceutically acceptable salt or/and polymorph thereof. In some embodiments of the present invention, the pterin Formula I is 5,6,7,8-tetrahydrobiopterin radical cation or a pharmaceutically acceptable salt or/and polymorph thereof. In some embodiments of the present invention, the pterin Formula I is L-threo-7,8-dihydrobiopterin or a pharmaceutically acceptable salt or/and polymorph thereof. In some embodiments of the present invention, the pterin Formula I is (6R)-L-threo-tetrahydrobiopterin or a pharmaceutically acceptable salt or/and polymorph thereof. In some embodiments of the present invention, the pterin Formula I is 7,8-dihydrobiopterin or a pharmaceutically acceptable salt or/and polymorph thereof. In some embodiments of the present invention, the pterin Formula I is L-erythro-5,6,7,8-tetrahydrobiopterin or a pharmaceutically acceptable salt or/and polymorph thereof. In some embodiments of the present invention, the pterin Formula I is 7,8-dihydromonapterin or a pharmaceutically acceptable salt or/and polymorph thereof. In some embodiments of the present invention, the pterin Formula I is 6-methyl-7-oxo-8-(1-D-ribityl) lumazine (2-hydroxy tautomer) (1-) or a pharmaceutically acceptable salt or/and polymorph thereof. In some embodiments of the present invention, the pterin Formula I is 6-methyl-7-oxo-8-(1-D-ribityl) lumazine (2-hydroxy tautomer) or a pharmaceutically acceptable salt or/and polymorph thereof. In some embodiments of the present invention, the pterin Formula I is 2-amino-6-[(1R)-1,2-dihydroxypropyl]-7,8-dihydro-1H-pteridin-4-one or a pharmaceutically acceptable salt or/and polymorph thereof. In some embodiments of the present invention, the pterin Formula I is 2-amino-6-[(1R)-1,2-dihydroxypropyl]-5,6,7,8-tetrahydro-1H-pteridin-4-one or a pharmaceutically acceptable salt or/and polymorph thereof. In some embodiments of the present invention, the pterin Formula I is Fosdenopterin. In some embodiments of the present invention, the pterin Formula I is tetrahydromonapterin. In some embodiments of the present invention, the pterin Formula I is D-erythro-7,8-dihydrobiopterin. In some embodiments, the 5,6,7,8-tetrahydrobiopterins or salts thereof is a 6R-(L-erythro)-5,6,7,8-tetrahydrobiopterin, also known as sapropterin or pharmaceutically acceptable salt or/and polymorph thereof. For various embodiments, the Sapropterin is Sapropterin hydrochloride or dihydrochloride.

In various embodiments, the pterin Formula I may be selected from the list consisting of: a pterin Formula (Ia), a pterin Formula (Ib), a pterin Formula (Ic), a pterin Formula (Id), sapropterin or a pharmaceutically acceptable salt or polymorph thereof. In some embodiments, the pterin Formula I may be a pterin Formula (Ia) or pharmaceutically acceptable salt or polymorph thereof. In some embodiments, the pterin Formula I may be a pterin Formula (Ib) or pharmaceutically acceptable salt or polymorph thereof. In some embodiments, the pterin Formula I may be a pterin Formula (Ic) or pharmaceutically acceptable salt or polymorph thereof. In some embodiments, the pterin Formula I may be a pterin Formula (Id) or pharmaceutically acceptable salt or polymorph thereof. In some embodiments, the pterin Formula I may be a sapropterin or pharmaceutically acceptable salt or polymorph thereof to the subject. In another embodiments, pterin Formula I is selected from a list consisting of: (6R)-6-lactoyl-5,6,7,8-tetrahydropterin, (6R)-5,6,7,8-tetrahydrobiopterin, 5,6,7,8-tetrahydrobiopterin, sepiapterin, 6-Methyltetrahydropterin, Hydroxysepiapterin, 7,8-dihydroneopterin, 6-lactoyl-5,6,7,8-tetrahydropterin, tetrahydroneopterin, leucopterin (keto form), 1-hydroxy-2-Oxopropyl tetrahydropterin, 6,7-Dimethyltetrahydropterin, 2-amino-5,6,7,8-tetrahydroxy-6-(1,2,3-trihydroxypropyl)-3,7-dihydropteridin-4-one, L-erythro-7,8-dihydrobiopterin, 2-amino-6-(hydroxymethyl)-7,8-dihydropteridin-4-one, tetrahydrodictyopterin, sapropterin, fosdenopterin, 5,6,7,8-tetrahydropterin-6-carboxylate, 5,6,7,8-tetrahydropterin-6-carboxylic acid, 5,6,7,8-tetrahydrobiopterin, radical cation, L-threo-7,8-dihydrobiopterin, (6R)-L-threo-tetrahydrobiopterin, 7,8-dihydrobiopterin, L-erythro-5,6,7,8-tetrahydrobiopterin, 7,8-dihydromonapterin, 6-methyl-7-oxo-8-(1-D-ribityl) lumazine (2-hydroxy tautomer) (1-), 6-methyl-7-oxo-8-(1-D-ribityl) lumazine (2-hydroxy tautomer), 2-amino-6-[(1R)-1,2-dihydroxypropyl]-7,8-dihydro-1H-pteridin-4-one, 2-amino-6-[(1R)-1,2-dihydroxypropyl]-5,6,7,8-tetrahydro-1H-pteridin-4-one, (6R)-L-erythro-5,6,7,8-tetrahydrobiopterin (BH4), (6R,S)-5,6,7,8-tetrahydrobiopterin, 1′,2′-diacetyl-5,6,7,8-tetrahydrobiopterin, 6-methyl-5,6,7,8-tetrahydropterin, 6-hydroxymethyl-5,6,7,8-tetrahydropterin, 6-phenyl-5,6,7,8-tetrahydropterin, tetrahydromonapterin, D-erythro-7,8-dihydrobiopterin, or pharmaceutically acceptable salt or/and polymorph thereof.

In aspects and embodiments of the present invention, the pterin is a known precursor, or functional equivalent of Formula I (Formula Ia, Ib, Ic or Id).

In various embodiments, administering of pterin Formula I may be separately, sequentially, or simultaneously with one or more aneurysm inhibitors. In some embodiments, the pterin Formula I may be concomitantly administered in combination with one or more aneurysm inhibitors. Examples of aneurysm inhibitors may be any known in the art. Examples include but are not limited to a group consisting of a beta blocker, a calcium channel blocker, an angiotensin II receptor blocker, a statin, and combinations thereof. The beta blockers employed may include but are not limited to acebutolol, atenolol, betaxolol, bisoprolol, carteolol, labetalol, metoprolol, nadolol, nebivolol, penbutolol, pindolol, propranolol, sotanol, timolol, and combinations thereof. The calcium channel blocker may be selected from a group consisting of amlodipine, beprifil, diltiazem, felodipine, isradipine, nicardipine, nifedipine, nisoldipine, verapamil, and combinations thereof. The angiotensin II receptor blocker may be selected from a group consisting of azilsartan, candesartan, eprosartan, irbesartan, losartan, olmesartan, termisartan, valsartan, and combinations thereof. The statin may be selected from a group consisting of atorvastatin, fluvastatin, lovastatin, pitavastatin, pravastatin, rosuvastatin, simvastatin, and combinations thereof.

In another aspect, there is provided a method for treating a subset of subjects diagnosed with or in a risk category for developing a vascular disease, comprising: (a) measuring F-actin and G-actin in a fraction of a biological sample from a subject; and (b) contingent upon the F-actin to G-actin ratio being reduced compared to that of a reference sample, administering a therapeutically effective amount of an Formula I to the subject.

The present disclosure, in accordance with aspects and embodiments herein, may include a detection kit. The kit could include all components necessary for performing any one or more of the following assays: primer extension assay such as allele-specific primer extension assays; nucleotide incorporation assay such as allele-specific nucleotide incorporation assays such as single base extension assays; oligonucleotide hybridization assay such as allele-specific oligonucleotide hybridization assays (e.g., oligonucleotide ligation assays); a 5′ nuclease assay; an assay employing molecular beacons; DNA sequencing, genetic bit analysis (GBA) and an oligonucleotide ligation assay. The kit may also include specific primers, oligonucleotide sequences, molecular beacon, or GBA which are specific to the genetic variations listed in the present invention.

In another aspect, there is provided a kit comprising components for detecting in a biological sample obtained from a subject, a presence of one or more genetic variations in an actin alpha 2 (ACTA2) gene, wherein the variation is a minor allele of a single nucleotide polymorphism (SNP) selected from a list consisting of: rs112602953, rs112901682, rs121434526, rs121434527, rs121434528, rs387906592, rs397515325, rs397516683, rs397516685, rs727502878, rs746972765, rs772862676, rs794728019, rs794728021, rs794728025, rs794728029, rs869025352, rs886038852, rs886038978, rs886039303, rs1057521105, rs1060500134, rs1064793016, rs1254836237, rs1554841843 and rs150547139.

In another aspect, there is provided a kit comprising components for detecting in a biological sample obtained from a subject, a presence of one or more genetic variations in an actin alpha 2 (ACTA2) gene, and further comprising a formulation of Formula I.

In another aspect of the present invention, the pterin Formula I is used in the manufacture of a medicament for treating subjects diagnosed with or in a risk category for developing an aneurysm. In aspects and embodiments of the present invention, the pterin Formula I is used in the manufacture of a medicament for treating subjects diagnosed with or in a risk category for developing an aortic aneurysm. In aspects and embodiments of the present invention, the pterin Formula I is used in the manufacture of a medicament for treating subjects diagnosed with or in a risk category for developing an aortic aneurysm and further having a genetic variation in the Actin alpha 2 (ACTA2) gene. In aspects and embodiments of the present invention, the pterin Formula I is used in the manufacture of a medicament for treating subjects diagnosed with or in a risk category for developing an aortic aneurysm and further having a genetic variation in the Actin alpha 2 (ACTA2) gene wherein the variation is the presence of a minor allele of a single nucleotide polymorphism (SNP) selected from the list consisting of: rs112602953, rs112901682, rs121434526, rs121434527, rs121434528, rs387906592, rs397515325, rs397516683, rs397516685, rs727502878, rs746972765, rs772862676, rs794728019, rs794728021, rs794728025, rs794728029, rs869025352, rs886038852, rs886038978, rs886039303, rs1057521105, rs1060500134, rs1064793016, rs1254836237, rs1554841843 and rs150547139. Specifically, the genetic variation is the presence of a minor allele of at least one single nucleotide polymorphism (SNP) is selected from the list consisting of: rs112602953, rs121434526, rs387906592, rs121434528.

In another aspect, there is provided a method for predicting sensitivity to treatment or indicative of a propensity for an increased likelihood of pharmacological effectiveness or benefit of treatment of pterin Formula (I) in a subject diagnosed with or in a risk category for developing vascular diseases associated with a genetic variation in ACTA2 such as premature onset coronary artery disease, premature ischemic stroke, Moyamoya disease and aortic aneurysms comprising identifying a genetic variation in the Actin alpha 2 (ACTA2) gene. In some embodiments, the genetic variation in the Actin Alpha 2 gene is the presence of a minor allele of at least one single nucleotide polymorphism (SNP) selected from the list consisting of: rs112602953, rs112901682, rs121434526, rs121434527, rs121434528, rs387906592, rs397515325, rs397516683, rs397516685, rs727502878, rs 746972765, rs772862676, rs794728019, rs794728021, rs794728025, rs794728029, rs869025352, rs886038852, rs886038978, rs886039303, rs1057521105, rs1060500134, rs1064793016, rs1254836237, rs 1554841843 and rs150547139. Specifically, the genetic variation may be the presence of a minor allele of at least one single nucleotide polymorphism (SNP) selected from the list consisting of: rs112602953, rs121434526, rs387906592, rs121434528.

In another aspect, there is provided a method for manufacturing a formulation comprising pterin Formula I for treatment of a subset of subjects diagnosed with or in a risk category for developing a vascular disease comprising: packaging the pterin formulation with instructions to administer the formulation to a subject selected based on detecting in a biological sample of the subject of one or more nucleotide substitutions selected from: a substitution that results in an amino acid change consisting of a substitution of arginine (R) for histidine (H) at position 258 (R258H) or a histidine (H) allele of a single nucleotide polymorphism (SNP) of rs121434527; a substitution that results in an amino acid change consisting of a substitution of methionine (M) for valine (V) at position 49 (M49V) or a valine (V) allele of a single nucleotide polymorphism (SNP) of rs397515325; a substitution that results in an amino acid change consisting of a substitution of alanine (A) for valine (V) at position 140 (A140V) or a valine (V) allele of a single nucleotide polymorphism (SNP) of rs397516683; a substitution that results in an amino acid change consisting of a substitution of arginine (R) for glutamine (Q) at position 212 (R212Q) or a glutamine (Q) allele of a single nucleotide polymorphism (SNP) of rs397516685; a substitution that results in an amino acid change consisting of a substitution of lysine (K) for asparagine (N) at position 240 (K240N) or a asparagine (N) allele of a single nucleotide polymorphism (SNP) of rs727502878; a substitution that results in an amino acid change consisting of a substitution of arginine (R) for histidine (H) at position 198 (R198H) or a histidine (H) allele of a single nucleotide polymorphism (SNP) of rs746972765; a substitution that results in an amino acid change consisting of a substitution of arginine (R) for cysteine (C) at position 198 (R198C) or a cysteine (C) allele of a single nucleotide polymorphism (SNP) of rs772862676; a substitution that results in an amino acid change consisting of a substitution of methionine (M) for valine (V) at position 1 (M1V) or a valine (V) allele of a single nucleotide polymorphism (SNP) of rs794728019; a substitution that results in an amino acid change consisting of a substitution of arginine (R) for histidine (H) at position 39 (R39H) or a histidine (H) allele of a single nucleotide polymorphism (SNP) of rs794728021; a substitution that results in an amino acid change consisting of a substitution of arginine (R) for histidine (H) at position 149 (R149H) or a histidine (H) allele of a single nucleotide polymorphism (SNP) of rs794728025; a substitution that results in an amino acid change consisting of a substitution of arginine (R) for leucine (L) at position 149 (R149L) or a leucine (L) allele of a single nucleotide polymorphism (SNP) of rs794728025; a substitution that results in an amino acid change consisting of a substitution of glycine (G) for glutamic acid (E) at position 270 (G270E) or a glutamic acid (E) allele of a single nucleotide polymorphism (SNP) of rs794728029; a substitution that results in an amino acid change consisting of a substitution of methionine (M) for lysine (K) or threonine (T) at position 49 (M49K or M49T) or alternatively, a lysine (K) or threonine (T) allele of a single nucleotide polymorphism (SNP) of rs869025352; a substitution that results in an amino acid change consisting of a substitution of arginine (R) for cysteine (C) or serine(S) at position 256 (R256C or R256S) or alternatively, a cysteine (C) or serine(S) allele of a single nucleotide polymorphism (SNP) of rs886038852; a substitution that results in an amino acid change consisting of a substitution of arginine (R) for cysteine (C) or serine(S) at position 179 (R179C or R179S) or alternatively, a cysteine (C) or serine(S) allele of a single nucleotide polymorphism (SNP) of rs886039303; a substitution that results in an amino acid change consisting of a substitution of arginine (R) for glutamine (Q) at position 185 (R185Q) or a glutamine (Q) allele of a single nucleotide polymorphism (SNP) of rs1057521105; a substitution that results in an amino acid change consisting of a substitution of proline (P) for leucine (L) at position 72 (P72L) or a leucine (L) allele of a single nucleotide polymorphism (SNP) of rs1060500134; a substitution that results in an amino acid change consisting of a substitution of glycine (G) for serine(S) at position 76 (G76S) or a serine(S) allele of a single nucleotide polymorphism (SNP) of rs1064793016; a substitution that results in an amino acid change consisting of a substitution of aspartic acid (D) for glutamic acid (E) at position 82 (D82E) or alternatively, a glutamic acid (E) allele of a single nucleotide polymorphism (SNP) of rs1254836237; a substitution that results in an amino acid change consisting of a substitution of methionine (M) for arginine (R) at position 46 (M46R) or alternatively, an arginine (R) allele of a single nucleotide polymorphism (SNP) of rs1554841843; a substitution that results in an amino acid change consisting of a substitution of aspartic acid (D) for glutamic acid (E) at position 58 (D58E) or a glutamic acid (E) allele of a single nucleotide polymorphism (SNP) of rs 150547139; a substitution that results in an amino acid change consisting of a substitution of arginine (R) for proline (P) at position 118 (R118P) or a proline (P) allele of a single nucleotide polymorphism SNP of rs112602953; a substitution that results in an amino acid change consisting of a substitution of arginine for glutamine at position 118 (R118Q) or a glutamine (Q) allele of a single nucleotide polymorphism SNP of rs112602953; a substitution that results in an amino acid change consisting of a substitution of arginine for cysteine at position 149 (R149C) or a cysteine (C) allele of a single nucleotide polymorphism SNP of rs121434526; a substitution that results in an amino acid change consisting of a substitution of arginine for histidine at position 179 (R179H) or a histidine (H) allele of a single nucleotide polymorphism SNP of rs387906592; and a substitution that results in an amino acid change consisting of a substitution of arginine for cysteine at position 258 (R258C) or a cysteine (C) allele of a single nucleotide polymorphism SNP of rs121434528.

In another aspect, there is provided a pterin Formula I or a pharmaceutically acceptable salt or polymorph thereof, for use in the manufacture of a formulation for the treatment of a subset of subjects diagnosed with or in a risk category for developing a vascular disease, wherein the subject population is selected based on detection in a biological sample of the presence of one or more nucleotide substitutions selected from: a substitution that results in an amino acid change consisting of a substitution of arginine (R) for histidine (H) at position 258 (R258H) or a histidine (H) allele of a single nucleotide polymorphism (SNP) of rs121434527; a substitution that results in an amino acid change consisting of a substitution of methionine (M) for valine (V) at position 49 (M49V) or a valine (V) allele of a single nucleotide polymorphism (SNP) of rs397515325; a substitution that results in an amino acid change consisting of a substitution of alanine (A) for valine (V) at position 140 (A140V) or a valine (V) allele of a single nucleotide polymorphism (SNP) of rs397516683; a substitution that results in an amino acid change consisting of a substitution of arginine (R) for glutamine (Q) at position 212 (R212Q) or a glutamine (Q) allele of a single nucleotide polymorphism (SNP) of rs397516685; a substitution that results in an amino acid change consisting of a substitution of lysine (K) for asparagine (N) at position 240 (K240N) or a asparagine (N) allele of a single nucleotide polymorphism (SNP) of rs727502878; a substitution that results in an amino acid change consisting of a substitution of arginine (R) for histidine (H) at position 198 (R198H) or a histidine (H) allele of a single nucleotide polymorphism (SNP) of rs746972765; a substitution that results in an amino acid change consisting of a substitution of arginine (R) for cysteine (C) at position 198 (R198C) or a cysteine (C) allele of a single nucleotide polymorphism (SNP) of rs772862676; a substitution that results in an amino acid change consisting of a substitution of methionine (M) for valine (V) at position 1 (M1V) or a valine (V) allele of a single nucleotide polymorphism (SNP) of rs794728019; a substitution that results in an amino acid change consisting of a substitution of arginine (R) for histidine (H) at position 39 (R39H) or a histidine (H) allele of a single nucleotide polymorphism (SNP) of rs794728021; a substitution that results in an amino acid change consisting of a substitution of arginine (R) for histidine (H) at position 149 (R149H) or a histidine (H) allele of a single nucleotide polymorphism (SNP) of rs794728025; a substitution that results in an amino acid change consisting of a substitution of arginine (R) for leucine (L) at position 149 (R149L) or a leucine (L) allele of a single nucleotide polymorphism (SNP) of rs794728025; a substitution that results in an amino acid change consisting of a substitution of glycine (G) for glutamic acid (E) at position 270 (G270E) or a glutamic acid (E) allele of a single nucleotide polymorphism (SNP) of rs794728029; a substitution that results in an amino acid change consisting of a substitution of methionine (M) for lysine (K) or threonine (T) at position 49 (M49K or M49T) or alternatively, a lysine (K) or threonine (T) allele of a single nucleotide polymorphism (SNP) of rs869025352; a substitution that results in an amino acid change consisting of a substitution of arginine (R) for cysteine (C) or serine(S) at position 256 (R256C or R256S) or alternatively, a cysteine (C) or serine(S) allele of a single nucleotide polymorphism (SNP) of rs886038852; a substitution that results in an amino acid change consisting of a substitution of arginine (R) for cysteine (C) or serine(S) at position 179 (R179C or R179S) or alternatively, a cysteine (C) or serine(S) allele of a single nucleotide polymorphism (SNP) of rs886039303; a substitution that results in an amino acid change consisting of a substitution of arginine (R) for glutamine (Q) at position 185 (R185Q) or a glutamine (Q) allele of a single nucleotide polymorphism (SNP) of rs1057521105; a substitution that results in an amino acid change consisting of a substitution of proline (P) for leucine (L) at position 72 (P72L) or a leucine (L) allele of a single nucleotide polymorphism (SNP) of rs1060500134; a substitution that results in an amino acid change consisting of a substitution of glycine (G) for serine(S) at position 76 (G76S) or a serine(S) allele of a single nucleotide polymorphism (SNP) of rs1064793016; a substitution that results in an amino acid change consisting of a substitution of aspartic acid (D) for glutamic acid (E) at position 82 (D82E) or alternatively, a glutamic acid (E) allele of a single nucleotide polymorphism (SNP) of rs1254836237; a substitution that results in an amino acid change consisting of a substitution of methionine (M) for arginine (R) at position 46 (M46R) or alternatively, an arginine (R) allele of a single nucleotide polymorphism (SNP) of rs1554841843; a substitution that results in an amino acid change consisting of a substitution of aspartic acid (D) for glutamic acid (E) at position 58 (D58E) or a glutamic acid (E) allele of a single nucleotide polymorphism (SNP) of rs 150547139; a substitution that results in an amino acid change consisting of a substitution of arginine (R) for proline (P) at position 118 (R118P) or a proline (P) allele of a single nucleotide polymorphism SNP of rs112602953; a substitution that results in an amino acid change consisting of a substitution of arginine (R) for proline (P) at position 118 (R118P) or a proline (P) allele of a single nucleotide polymorphism SNP of rs112602953; a substitution that results in an amino acid change consisting of a substitution of arginine for glutamine at position 118 (R118Q) or a glutamine (Q) allele of a single nucleotide polymorphism SNP of rs112602953; a substitution that results in an amino acid change consisting of a substitution of arginine for cysteine at position 149 (R149C) or a cysteine (C) allele of a single nucleotide polymorphism SNP of rs121434526; a substitution that results in an amino acid change consisting of a substitution of arginine for histidine at position 179 (R179H) or a histidine (H) allele of a single nucleotide polymorphism SNP of rs387906592; and a substitution that results in an amino acid change consisting of a substitution of arginine for cysteine at position 258 (R258C) or a cysteine (C) allele of a single nucleotide polymorphism SNP of rs121434528.

In some embodiments, there is provided a pterin of Formula I or a pharmaceutically acceptable salt or polymorph thereof, for use in the manufacture of a formulation wherein the pterin is selected from the list consisting of: (6R)-6-lactoyl-5,6,7,8-tetrahydropterin, (6R)-5,6,7,8-tetrahydrobiopterin, 5,6,7,8-tetrahydrobiopterin, sepiapterin, 6-Methyltetrahydropterin, Hydroxysepiapterin, 7,8-dihydroneopterin, 6-lactoyl-5,6,7,8-tetrahydropterin, Tetrahydroneopterin, leucopterin (keto form), 1-hydroxy-2-Oxopropyl tetrahydropterin, 6,7-Dimethyltetrahydropterin, 2-amino-5,6,7,8-tetrahydroxy-6-(1,2,3-trihydroxypropyl)-3,7-dihydropteridin-4-one, Fosdenopterin, L-erythro-7,8-dihydrobiopterin, 2-amino-6-(hydroxymethyl)-7,8-dihydropteridin-4-one, tetrahydrodictyopterin, sapropterin, 5,6,7,8-tetrahydropterin-6-carboxylate, 5,6,7,8-tetrahydropterin-6-carboxylic acid, 5,6,7,8-tetrahydrobiopterin, radical cation, L-threo-7,8-dihydrobiopterin, (6R)-L-threo-tetrahydrobiopterin, 7,8-dihydrobiopterin, L-erythro-5,6,7,8-tetrahydrobiopterin, 7,8-dihydromonapterin, 6-methyl-7-oxo-8-(1-D-ribityl) lumazine (2-hydroxy tautomer) (1-), 6-methyl-7-oxo-8-(1-D-ribityl) lumazine (2-hydroxy tautomer), 2-amino-6-[(1R)-1,2-dihydroxypropyl]-7,8-dihydro-1H-pteridin-4-one, 2-amino-6-[(1R)-1,2-dihydroxypropyl]-5,6,7,8-tetrahydro-1H-pteridin-4-one, (6R)-L-erythro-5,6,7,8-tetrahydrobiopterin (BH4), (6R,S)-5,6,7,8-tetrahydrobiopterin, 1′,2′-diacetyl-5,6,7,8-tetrahydrobiopterin, 6-methyl-5,6,7,8-tetrahydropterin, 6-hydroxymethyl-5,6,7,8-tetrahydropterin, 6-phenyl-5,6,7,8-tetrahydropterin or pharmaceutically acceptable salt or/and polymorph thereof.

In some embodiments, the formulation comprises an amount of pterin Formula I sufficient to cause any one or more of the following in the subject subsequent to treatment: an increase in functional F-actin; an increase in the ratio between F-actin to G-actin; a decrease in functional G-actin; improvement in edema and an increase in contractile force in aortic vascular smooth muscle cells.

In various embodiments, the detection in a biological sample comprises carrying out a process selected from: a primer extension assay; nucleotide incorporation assay, oligonucleotide hybridization assay, a 5′ nuclease assay, an assay employing molecular beacons, DNA sequencing, genetic bit analysis (GBA) and an oligonucleotide ligation assay.

In aspects and embodiments of the present invention, any vascular diseases previously associated or not yet associated with genetic variation in ACTA2 may make a candidate relevant for treatment. Diseases which have not yet been associated with ACTA2 genetic variation are also included in the present invention, especially in the context of prophylactic treatment. Patients having vascular diseases which have already been identified as being associated with an ACTA2 genetic variation as well as patients having diseases not yet associated with vascular diseases, in the context of this invention, extend to any ACTA2 genetic variation which would not affect the 3-dimensional orientation of any one or more specific amino acids, namely 75 (His), 78 (Ile), 193 (Lys), and 196 (Thr). In some embodiments, any SNP of the genetic locus or loci of a gene ACTA2 which is not affected by the 3-dimensional orientation of any one or more of the following amino acids: 75 (His), 78 (Ile), 193 (Lys), and 196 (Thr) could be included in the selection of patients for treatment.

In various embodiments, the vascular disease is selected from the list consisting of: premature onset coronary artery disease, premature ischemic strokes, Moyamoya disease and especially aortic aneurysms. In some embodiments, the aneurysm is an aortic aneurysm. For example, the aortic aneurysm may be a descending aortic aneurysm, an ascending aortic aneurysm, and/or an abdominal aorticaneurysm. In some embodiments, the ascending aortic aneurysm is an ascending thoracic aortic aneurysm.

In some embodiments, the formulation further comprises one or more aneurysm inhibitors. In some embodiments, the aneurysm inhibitor is selected from the group consisting of a beta blocker, a calcium channel blocker, an angiotensin II receptor blocker, a statin, and combinations thereof. The beta blocker may be selected from the group consisting of acebutolol, atenolol, betaxolol, bisoprolol, carteolol, labetalol, metoprolol, nadolol, nebivolol, penbutolol, pindolol, propranolol, sotanol, timolol, and combinations thereof. The calcium channel blocker is selected from the group consisting of amlodipine, beprifil, diltiazem, felodipine, isradipine, nicardipine, nifedipine, nisoldipine, verapamil, and combinations thereof. The angiotensin II receptor blocker may be selected from the group consisting of azilsartan, candesartan, eprosartan, irbesartan, losartan, olmesartan, termisartan, valsartan, and combinations thereof. The statin may be selected from the group consisting of atorvastatin, fluvastatin, lovastatin, pitavastatin, pravastatin, rosuvastatin, simvastatin, and combinations thereof.

### BRIEF DESCRIPTION OF THE FIGURES

Aspects of the present disclosure are best understood from the following detailed description when read with the accompanying figures. It is emphasized that, under standard practice in the industry, various features are not drawn to scale. The dimensions of the various features may be arbitrarily increased or reduced for clarity of discussion. In addition, the present disclosure may repeat reference numerals and/or letters in multiple examples.

FIG. 1 is a table listing the different mutations in ACTA2 which were identified as candidates for rescue treatment by Formula I (e.g., Sapropterin) and related compounds.

FIG. 2 and FIG. 3 represents the molecules of the present invention which are capable of treating or compensating for ACTA2 mutant actin structures.

FIG. 4 is a model demonstrating the interaction between sapropterin and ACTA2 mutants.

FIG. 5 presents quantitative results of edema in a zebrafish model of human ACTA2 R149C.

### DETAILED DESCRIPTION

The present disclosure describes products and methods involving a pterin of Formula I (e.g., Formula Ia, Ib or Ic) or more specifically tetrahydrobiopterin (e.g., Sapropterin) for use in a subset of a patient population at risk for developing or diagnosed with a vascular disease such as premature onset coronary artery disease, premature ischemic strokes, Moyamoya disease or an aortic aneurysm. More specifically, the present disclosure describes products and methods involving a pterin of Formula I (e.g., Formula Ia, Ib, Ic or Id) or more specifically tetrahydrobiopterin (also known as sapropterin) for use in a subset of a patient population at risk for developing or diagnosed with a vascular disease involving ACTA2 mutations.

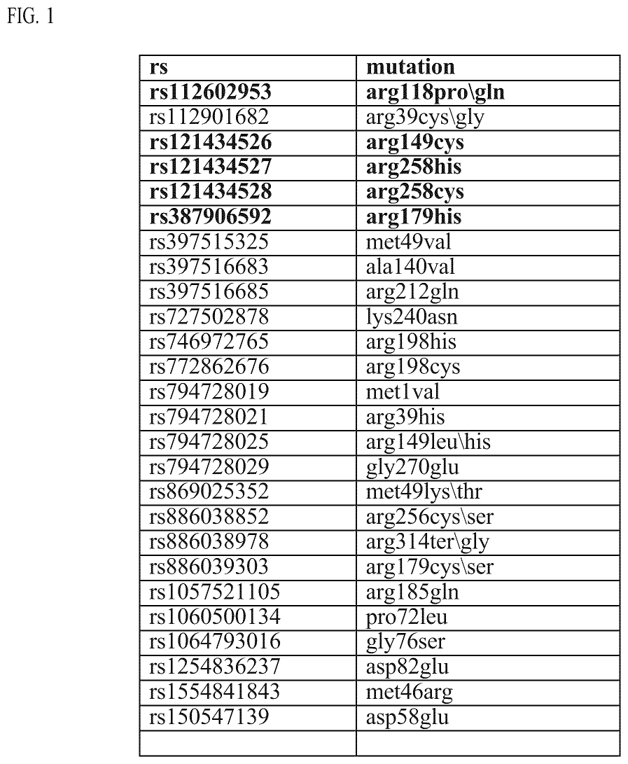

Recent advances in electron cryomicroscopy has led to high resolution (3.7-4.7 Å) of structures of the actin filament. The inventors have discovered, using molecular dynamic simulation using two to four monomers, the presence of an increase ininter-subunit distances between two acta subunits (across the strand, between unit G and H) in the case of a specific list of ACTA2 genetic mutations (substitutions) presented in bold in FIG. 1. These four potentially deleterious substitutions in the ACTA2 amino acid sequence (R118P/Q, R149C, R179H, R259C) are frequently seen in patients with familial ascending thoracic aortic aneurysms. Each of these mutations have more than a 2-angstrom difference in distance compared with those measured in the normal actin fiber. Other variations in ACTA2 disclosed in FIG. 1 also involve an increased inter-subunit distance.

The SNPs identified herein in FIG. 1 provide targets for development of therapeutic agents for use in the diagnosis and treatment of genetically identified subjects, including diagnosis and targeted treatment for patient subpopulations exhibiting the distinct genetic signature comprising one or more of the SNPs of the present invention. For example, in one aspect, the genes containing the genetic variations identified herein, and the nucleic acid (e.g., DNA or RNA) associated with these genes, and proteins encoded by these genes, can be used as targets for the development of therapeutic agents (e.g., small molecule compounds, antibodies, antisense/RNAi agents, etc.) or used directly as therapeutic agents (e.g., therapeutic proteins, etc.) for the treatment of vascular diseases.

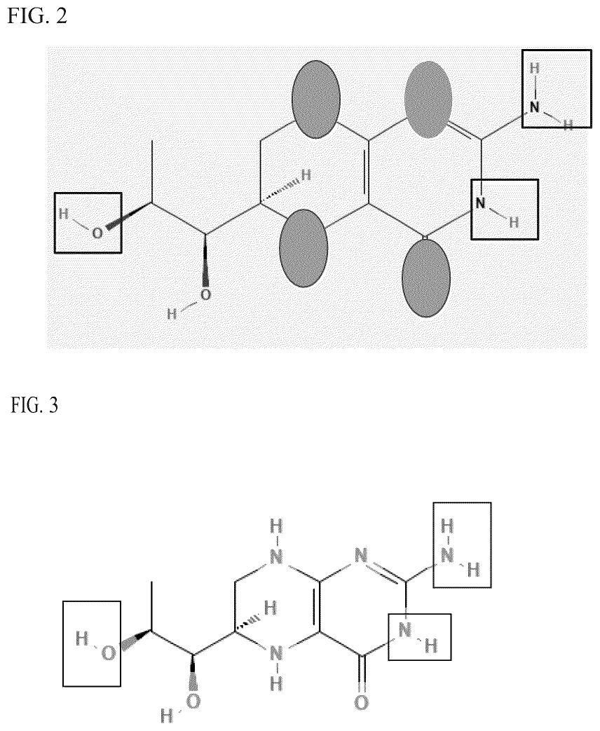

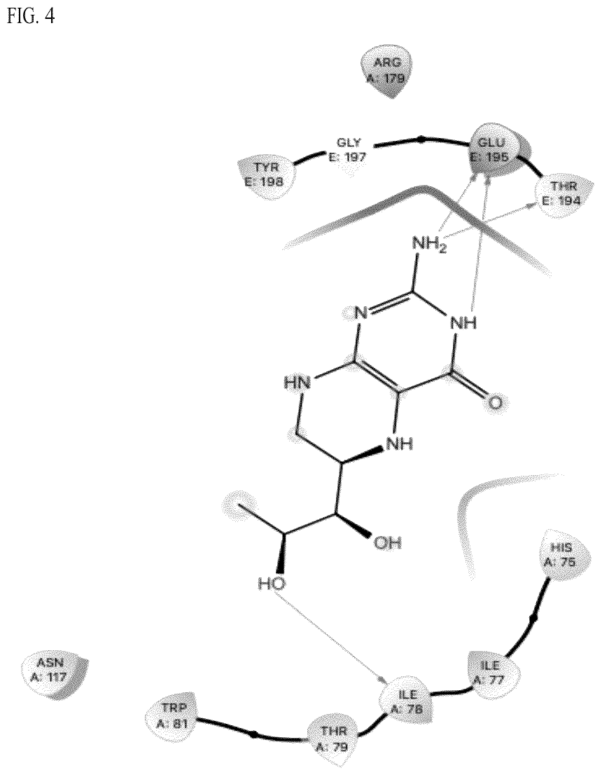

Next, the inventors screened tens of thousands of molecules to identify that the structures presented in FIG. 2 of a pterin Formula I (e.g., Formula Ia, Ib, Ic, Id) or more specifically, sapropterin in FIG. 3, can return the native structure to the actinA2 as demonstrated in FIG. 4. In FIGS. 2 and 3, the molecules with a box represent those which are proposed to interact with actin, while those blocked with a circle, represent those which only contribute to general positioning in space and therefore specific molecule can be less critical as long as the general structure in space is minimally affected. The pterin Formula I apparently binds in some form to amino acid residues HIS H: 75 and/or ILE A: 78 as well as ARG R: 179 and/or LYS K: 193 and/or THR T: 196 of the human ACTA2, despite the genetic variation, thereby compensating for dysfunctional activity. In some embodiments, the pterin derivative Formula I binds in some form to amino acid residues HIS H: 75 and/or ILE A: 78 as well as ARG R: 179 and/or LYS K: 193 and/or THR T: 196 of the human ACTA2, thereby compensating for dysfunctional activity. In some embodiments, the pterin derivative Formula I binds in some form to amino acid residues HIS H: 75 or ILE A: 78 as well as ARG R: 179 or LYS K: 193 or THR T: 196 of the human ACTA2, thereby compensating for dysfunctional activity of the ACTA2 genetic variation.

This discovery, for diseases without treatment to date, was further validated in a number of animal models for a couple of genetic variations. Based on the results in animal models, it is suggested that patients having, or at risk for developing vascular diseases and identified as having at least one ACTA2 mutation, may be sensitive or may indicate a propensity for an increased likelihood of pharmacological effectiveness or benefit of treatment by a pterin having Formula I. These subjects may be treated by a pterin of Formula (Ia), (Ib), (Ic), (Id) such as sapropterin. The list of ACTA2 genetic variations of relevance for treatment should not include any variation which substantially affects the predicted 3-dimensional orientation of HIS H: 75 or ILE A: 78 as well as ARG R: 179, LYS K: 193 or THR T: 196 in the resulting actin alpha 2 protein.

Thus, a pterin of Formula I, such as sapropterin, may be a means to mitigate or provide early intervention or treatment rather over traditional clinical evaluation and awaiting a lethal clinical event to occur. In some embodiments, it is provided to a fetus by administering to a pregnant adult thereby exposing the fetus during development. For example, early indicators may prompt genetic testing, which is common practice during pregnancy, and provide opportunity to influence development of the phenotype.

For purposes of interpreting this specification, the following definitions will apply and whenever appropriate, terms used in the singular will also include the plural and vice versa. As used in this specification and the appended claims, the singular terms “a,” an” and “the” include plural referents unless the context clearly dictates otherwise.

As used herein, “subject” or “patient” is a vertebrate. In certain embodiments, the vertebrate is a mammal. Mammals include, but are not limited to, primates and rodents. In certain embodiments, a mammal is a human. Illustratively, “subject”, as used herein, is meant to include human fetuses, infants, children, adolescents, adults, who are in utero, less than about 2 years of age, humans who are between about 2 years of age and 5 years of age, humans who are between about 5 and about 10 years of age, humans who are between about 10 and about 18 years of age, humans who are between about 18 and about 30 years of age, humans who are above 30 years of age, and humans who are over about 60 years of age.

The term “pterin” refers to all natural and unnatural stereoisomeric forms of pterin which have the following formula (Ia), Formula (Ib) or Formula (Ic) in free base or pharmaceutically acceptable salt form or/and polymorphs thereof.

The pterin derivative, as used herein in accordance with aspects and embodiments of the present invention, may have the following formula (Ia) in free base or pharmaceutically acceptable salt form or/and polymorphs thereof:

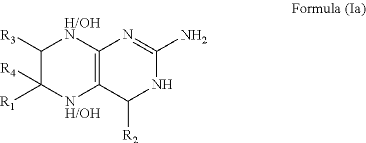

wherein
NH/OH represents an amine or hydroxylamine (i.e., nitrogen atom, further bound to a hydrogen or hydroxyl group),
R1 represents a hydrogen, alkyl (e.g., methy), hydroxyalkyl, hydroxyl, carbonyl, phosphate, ether or ketone group,
R2 represents an oxygen or amine group,
R3 represents an oxygen, hydrogen, methyl, or hydroxyl group and
R4 represents an oxygen, hydrogen, methyl, or hydroxyl group.

The pterin derivative, as used herein in accordance with aspects and embodiments of the present invention, may have the following formula (Ib) in free base or pharmaceutically acceptable salt form or/and polymorphs thereof:

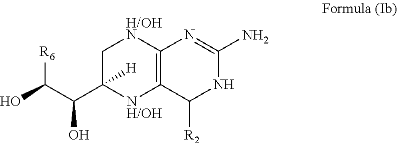

wherein
NH/OH represents an amine or hydroxylamine (i.e., nitrogen atom, further bound to a hydrogen or hydroxyl group),
O/OH represents a hydroxyl group, or an oxygen atom on a double bond,
R6 represents an alkyl (e.g., methyl), hydroxyalkyl, hydroxyl, carbonyl, phosphate, ketone, or ether, and
R2 represents an oxygen or amine group.

In Formula Ia and Ib (herein Formula I), R1 or R6 may be selected from the list consisting of the following: CH₂OH, CH₂CH₂OH, CH₃, CH₂CH₃, HCOHCH₂OH, CHOHCH₃, CHOHCH₂OH, COCH₃, OH, O, CH₂CH₂OH, CHOHCH₂P3010H4. In some embodiments, R2 is selected from the list consisting of the following: NH2, O or OH. Numerous combinations of R1 and R2 can also be envisioned.

The pterin derivative, as used herein in accordance with aspects and embodiments of the present invention, may have the following formula (Ic) in free base or pharmaceutically acceptable salt form or/and polymorphs thereof:

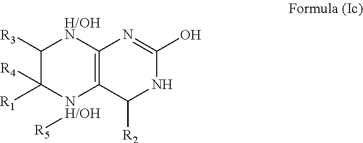

wherein
NH/OH represents an amine or hydroxylamine (i.e., nitrogen atom, further bound to a hydrogen or hydroxyl group),
R1 represents a hydrogen, alkyl (e.g., methy), hydroxyalkyl, hydroxyl, carbonyl, phosphate, ether or ketone group,
R2 represents an oxygen or hydroxyl group,
R3 represents an oxygen, hydrogen, methyl or hydroxyl group,
R4 represents an oxygen, hydrogen, methyl or hydroxyl group, and
R5 represents a hydroxyl or hydroxyl alcohol.

Other pterins, e.g., biopterins such as tetrahydrobiopterins may also be relevant. Molecules of the present invention include the following ones and pharmaceutically acceptable salts thereof: 6R)-6-lactoyl-5,6,7,8-tetrahydropterin, (6R)-5,6,7,8-tetrahydrobiopterin, 5,6,7,8-tetrahydrobiopterin, sepiapterin, 6-Methyltetrahydropterin, Hydroxysepiapterin, 7,8-dihydroneopterin, 6-lactoyl-5,6,7,8-tetrahydropterin, Tetrahydroneopterin, leucopterin (keto form), 1-hydroxy-2-Oxopropyl tetrahydropterin, 6,7-Dimethyltetrahydropterin, 2-amino-5,6,7,8-tetrahydroxy-6-(1,2,3-trihydroxypropyl)-3,7-dihydropteridin-4-one, Fosdenopterin, L-erythro-7,8-dihydrobiopterin, 2-amino-6-(hydroxymethyl)-7,8-dihydropteridin-4-one, tetrahydrodictyopterin, sapropterin, 5,6,7,8-tetrahydropterin-6-carboxylate, 5,6,7,8-tetrahydropterin-6-carboxylic acid, 5,6,7,8-tetrahydrobiopterin, radical cation, L-threo-7,8-dihydrobiopterin, (6R)-L-threo-tetrahydrobiopterin, 7,8-dihydrobiopterin, L-erythro-5,6,7,8-tetrahydrobiopterin, 7,8-dihydromonapterin, 6-methyl-7-oxo-8-(1-D-ribityl) lumazine (2-hydroxy tautomer) (1-), 6-methyl-7-oxo-8-(1-D-ribityl) lumazine (2-hydroxy tautomer), 2-amino-6-[(1R)-1,2-dihydroxypropyl]-7,8-dihydro-1H-pteridin-4-one, 2-amino-6-[(1R)-1,2-dihydroxypropyl]-5,6,7,8-tetrahydro-1H-pteridin-4-one, (6R)-L-erythro-5,6,7,8-tetrahydrobiopterin (BH4), (6R,S)-5,6,7,8-tetrahydrobiopterin, 1′,2′-diacetyl-5,6,7,8-tetrahydrobiopterin, 6-methyl-5,6,7,8-tetrahydropterin, 6-hydroxymethyl-5,6,7,8-tetrahydropterin, 6-phenyl-5,6,7,8-tetrahydropterin or pharmaceutically acceptable salt or/and polymorph thereof. In aspects and embodiments of the present invention, the pterin Formula I is a tetrahydrobiopterin or pharmaceutically acceptable salt or/and polymorph thereof.

In some embodiments, the term “tetrahydrobiopterin” refers to sepiapterin, a naturally occurring precursor of tetrahydrobiopterin displayed below. For example, the sepiapterin may be a crystalline form of sepiapterin free base or salts thereof as disclosed in U.S. Pat. Nos. 11,072,614, 11,130,760 or US20220081443.

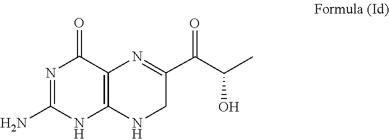 [SMILES: C[C@H](O)C(=O)C1=Nc2c([nH]c(N)nc2=O)NC1]

All compounds may be used as appropriate salts with pharmacologically non-toxic acids, including mineral acids such as hydrochloric acid, phosphoric acid, sulfuric acid, boric acid; and organic acids such as acetic acid, formic acid, maleic acid, fumaric acid, mesylic acid.

As used herein, “treating”, “treat” or “treatment” grammatical variations thereof, refers to clinical intervention in an attempt to alter the natural course of the individual or in some cases the aneurysm being treated, and can be performed before (, such as in “prophylactic treatment” for example in utero) or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and/or achieving remission or improved prognosis. In some embodiments, a method for treating may relate to a method of predicting sensitivity or indicative of a propensity for an increased likelihood of pharmacological effectiveness or benefit of treatment. In some embodiments, methods and compositions of the invention are useful in attempts to delay development of a disease or disorder. In some embodiments, the effectivity of a particular treatment may be measured by an increase in functional F-actin, an increase in the ratio between F-actin to G-actin, a decrease in functional G-actin or an increase in contractile force in aortic vascular smooth muscle cells.

A “subpopulation” or “patient subpopulation,” as used herein, refers to a patient subset characterized measurable and/or identifiable mutations that distinguishes the patient subset from others in the broader disease category to which it belongs.

The term “sample” or “biological sample”, as used herein, refers to a composition that is obtained or derived from a subject of interest that contains a genetic entity that is to be characterized and/or identified, for example using mutations detection methods. For example, the phrases “biological sample” refers to any sample obtained from a subject of interest that would be expected or is known to contain the genetic mutation that is to be characterized. Examples of samples include but are not limited to blood, solid tissue as from a fresh, frozen and/or preserved organ or tissue sample, biopsy, or aspirate; bodily fluids such as amniotic fluid, peritoneal fluid, or interstitial fluid; or cells from any time in gestation or development of the subject. The tissue sample may also be primary or cultured cells or cell lines.

As used herein, amino acid sequence of native ACTA2 has 377 amino acids.

42009 MW; 2D0543262DB35CA5 CRC64;
P62736; B2R8A4; P03996; P04108; Q6FI19;
SEQ1
MCEEEDSTALVCDNGSGLCK AGFAGDDAPR AVFPSIVGRP RHQGVMVGMG
QKDSYVGDEAQSKRGILTLK YPIEHGIITN WDDMEKIWHH SFYNELRVAP
EEHPTLLTEA PLNPKANREKMTQIMFETFN VPAMYVAIQA VLSLYASGRT
TGIVLDSGDG VTHNVPIYEG YALPHAIMRLDLAGRDLTDY LMKILTERGY
SFVTTAEREI VRDIKEKLCY VALDFENEMA TAASSSSLEKSYELPDGQVI
TIGNERFRCP ETLFQPSFIG MESAGIHETT YNSIMKCDID
IRKDLYANNVLSGGTTMYPG IADRMQKEIT ALAPSTMKIK IIAPPERKYS
VWIGGSILAS LSTFQQMWISKQEYDEAGPS IVHRKCF//

In a first aspect, the present invention provides specific nucleotide variations in ACTA2 which the inventors have now associated with potential treatment by pterin derivatives (i.e., Formula Ia, Ib, Ic, or sapropterin). Thus, in another aspect, there is provided a method for treating a subject having one or more genetic variations in an Actin alpha 2 (ACTA2) gene, comprising: administering a therapeutically effective amount of pterin Formula I (e.g., sapropterin) to a subject, contingent upon the presence of one or more specific genetic variations. These Actin alpha 2 (ACTA2) gene variations provide a basis for selecting relevant patient populations who may benefit from treatment. Benefit from treatment may include in this context, prevention, or decreased development and/or progression of actin-related diseases (i.e., diseases resulting from disturbance of the actin dynamic). Accordingly, the invention disclosed herein is useful in a variety of settings, e.g., in methods and compositions related to diagnosis and therapy of actin-related diseases (i.e., diseases resulting from disturbance of the actin dynamic).

The present invention discloses methods and products involving the selection for treatment of a patient population having genetic variation at a nucleotide position corresponding to the position of a SNP. The term “variation”, unless otherwise specified, refers to either a nucleotide variation or an amino acid variation.

The term “genetic variation” or “nucleotide variation” in ACTA2 or alternatively, “ActinA2 mutation” are used interchangeably herein and refer to a change in a nucleotide sequence (e.g., an insertion, deletion, inversion, or substitution of one or more nucleotides, such as a single nucleotide polymorphism (SNP)) relative to a reference sequence (e.g., a commonly found and/or wild-type sequence, and/or the sequence of a major allele). The term also encompasses the corresponding change in the complement of the nucleotide sequence, unless otherwise indicated.

The term “a genetic variation at a nucleotide position corresponding to the position of a SNP,” a nucleotide variation at a nucleotide position corresponding to the position of a SNP,” and grammatical variants thereof refer to a nucleotide variation in a polynucleotide sequence at the relative corresponding DNA position occupied by said SNP in the genome. The term also encompasses the corresponding variation in the complement of the nucleotide sequence, unless otherwise indicated.

A “single nucleotide polymorphism”, or “SNP”, refers to a single base position in DNA at which different alleles, or alternative nucleotides, exist in a population. The SNP position is usually preceded by and followed by highly conserved sequences of the allele (e.g., sequences that vary in less than 1/100 or 1/1000 members of the populations). An individual may be homozygous or heterozygous for an allele at each SNP position.

In various embodiments, the present invention relates to specific genetic variation in a subset of patients, wherein the one or more genetic variations include the presence of minor allele of at least one single nucleotide polymorphism (SNP) selected from the list consisting of: rs112602953, rs112901682, rs121434526, rs121434527, rs121434528, rs387906592, rs397515325, rs397516683, rs397516685, rs727502878, rs746972765, rs772862676, rs794728019, rs794728021, rs794728025, rs794728029, rs869025352, rs886038852, rs886038978, rs886039303, rs1057521105, rs1060500134, rs1064793016, rs1254836237, rs1554841843 and rs150547139, or more specifically rs112602953, rs112602953, rs121434526, rs387906592; and rs121434528.

In various embodiments of the present invention, the one or more genetic variations in the Actin alpha 2 (ACTA2) gene includes any SNP of the genetic locus or loci of a gene ACTA2 which does not substantially affect the 3-dimensional orientation of any one or more specific amino acids, namely 75 (His), 78 (Ile), 193 (Lys), 196 (Thr) and 78 (Ile). In some embodiments, any SNP of the genetic locus or loci of a gene ACTA2 which does not affect the 3-dimensional orientation of any one or more of the following amino acids: 75 (His) or LYS 78 (Ile) as well as 179 (Arg), 193 (Lys) or 196 (Thr). In some embodiments, any SNP of the genetic locus or loci of a gene ACTA2 which does not affect the 3-dimensional orientation of any one or more of the following amino acids: 75 (His), 78 (Ile), 193 (Lys) and 196 (Thr). Those skilled in the art using readily available computational tools can further determine additional SNPs of the ACTA2 gene which do not result in a significant shift in the 3-dimensional orientation of the listed amino acids. A significant shift may be defined as a shift of more than 3, more than 4, more than 5 or more than 5.5 Angstrom compared to the native conformation of the protein.

In accordance with aspects and embodiments of the disclosure, the ACTA2 mutation is a substitution. For example, the ACTA2 substitution may include a mutation which results in the distancing of specific amino acids a specific distance. In particular, the present invention relates to a specific set of mutations wherein the distance between amino acid R179 (chain A) and K191 (chain B) is greater than 7 angstroms in ACTA2. In another example, the present invention relates to a specific set of mutations wherein the distance between amino acid R179 (chain A) and K193 (chain B) is greater than 8.25 angstrom in ACTA2. In yet another example, the present invention relates to a specific set of mutations wherein the distance between amino acid G270 (chain A) and Q59 (chain B) is greater than 5 angstroms in ACTA2. In another example, the present invention relates to a specific set of mutations wherein the distance between amino acid M285 (chain A) and ILE267 (chain B) is greater than 5.95 angstrom in ACTA2.

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature. In addition, primers, oligonucleotides and polynucleotides employed in the present invention can be generated using standard techniques known in the art. Unless defined otherwise, technical and scientific terms used herein have the same meaning as commonly understood by one of ordinary skill in the art to which this invention belongs.

The present disclosure, in accordance with aspects and embodiments herein involves a method for treating a subject diagnosed with or in a risk category for developing a vascular disease. In a first step, the method includes detecting, in a biological sample obtained from the subject, the presence of one or more genetic variations in the Actin alpha 2 (ACTA2) gene. In some embodiments, the genetic variation is a minor allele of a single nucleotide polymorphism (SNP) selected from the list consisting of: rs112602953, rs112901682, rs121434526, rs121434527, rs121434528, rs387906592, rs397515325, rs397516683, rs397516685, rs727502878, rs746972765, rs772862676, rs794728019, rs794728021, rs794728025, rs794728029, rs869025352, rs886038852, rs886038978, rs886039303, rs1057521105, rs1060500134, rs1064793016, rs1254836237, rs1554841843 and rs150547139. In a second step, the method includes administering a therapeutically effective amount of pterin Formula I (e.g., Ia, Ib, Ic or Sapropterin) to the subject when the one or more variations are present. In some embodiments, the pterin is Formula I.

In some embodiments, the pterin is Formula Ia. In some embodiments, the pterin is Formula Ib. In some embodiments, the pterin is Formula Ic. In some embodiments, the pterin is Formula Id. In some embodiments, the pterin is tetrahydrobiopterin (also known as sapropterin)

Detecting in this context may involve any known method in the art. Detection of variations in target nucleic acid sequence may be accomplished by amplification or molecular cloning and sequencing of the target nucleic acids using techniques well known in the art. The term “amplification” refers to the process of producing one or more copies of a reference nucleic acid sequence or its complement. Amplification may be linear or exponential. The term “target sequence” or “target nucleic acid sequence” refers generally to a polynucleotide sequence of interest in which a nucleotide variation is suspected or known to reside, including copies of such target nucleic acid generated by amplification. Amplification may be desirable in certain instances, e.g., in order to obtain a desired amount of material for detecting variations. The amplicons may then be subjected to a detection method, such as those described below, to determine whether a variation is present in the amplicon.

The nucleic acid sequence of the amplified sequences can then be determined. A nucleic acid is said to be “derived from” a particular source if it is obtained directly from that source or if it is a copy of a nucleic acid found in that source such as a copy that result from amplification. Nucleic acid, as used herein, may be genomic DNA, RNA transcribed from genomic DNA, or cDNA generated from RNA. For example, where appropriate, the present invention may involve detecting an actin alpha variant in a biological sample. Detecting in this context may involve any known method in the art. Examples of known methods include but are not limited to a primer extension assay such as allele-specific primer extension assays; nucleotide incorporation assay such as allele-specific nucleotide incorporation assays such as single base extension assays; oligonucleotide hybridization assay such as allele-specific oligonucleotide hybridization assays (e.g., oligonucleotide ligation assays); a 5′ nuclease assay; an assay employing molecular beacons; DNA sequencing, genetic bit analysis (GBA) and an oligonucleotide ligation assay.

The term “detection” includes any means of detecting, including direct and indirect detection. Variations may be detected by certain methods known to those skilled in the art. Such methods include, but are not limited to, primer extension assay such as allele-specific primer extension assays; nucleotide incorporation assay such as allele-specific nucleotide incorporation assays such as single base extension assays; oligonucleotide hybridization assay such as allele-specific oligonucleotide hybridization assays (e.g., oligonucleotide ligation assays); a 5′ nuclease assay; an assay employing molecular beacons; DNA sequencing, genetic bit analysis (GBA) and an oligonucleotide ligation assay. Some of these methods will be expanded upon below.

In primer extension assays, an allele-specific primer is used wherein the 3′ terminal nucleotide of the primer is complementary to, that is, capable of specifically base-pairing with a particular variation in the target nucleic acid. If the particular variation is not present, an amplification product is not observed. The term “allele-specific primer” refers to an allele-specific oligonucleotide that is a primer. The term “primer” refers to a single stranded polynucleotide that is capable of hybridizing to a nucleic acid and allowing the polymerization of a complementary nucleic acid, generally by providing a free 3′OH group. The term “primer extension assay” refers to an assay in which nucleotides are added to a nucleic acid, resulting in a longer nucleic acid, or “extension product,” that is detected directly or indirectly. The nucleotides can be added to extend the 5′ or 3′ end of the nucleic acid. The term “allele-specific primer extension assay” refers to a primer extension assay in which an allele-specific primer is hybridized to a target nucleic acid and extended.

The term “allele-specific nucleotide incorporation assay” refers to a primer extension assay in which a primer is (a) hybridized to target nucleic acid at a region that is 3′ or 5′ of a nucleotide variation and (b) extended by a polymerase, thereby incorporating into the extension product a nucleotide that is complementary to the nucleotide variation. Examples include but are not limited to allele-specific PCR or ligation chain reaction (LCR).

The term “allele-specific oligonucleotide” refers to an oligonucleotide that hybridizes to a region of a target nucleic acid that comprises a nucleotide variation such as a substitution. “Allele-specific oligonucleotide hybridization” means that, when an allele-specific oligonucleotide is hybridized to its target nucleic acid, a nucleotide in the allele-specific oligonucleotide specifically base pairs with the nucleotide variation. An allele-specific oligonucleotide capable of allele-specific hybridization with respect to a particular nucleotide variation is said to be “specific for” that variation. The term “allele-specific oligonucleotide hybridization assay” refers to an assay in which (a) an allele-specific oligonucleotide is hybridized to a target nucleic acid and (b) hybridization is detected directly or indirectly.

The term “5′ nuclease assay” refers to an assay in which hybridization of an allele-specific oligonucleotide to a target nucleic acid allows for nucleolytic cleavage of the hybridized probe, resulting in a detectable signal. The word “signal” when used herein refers to a compound or composition which is conjugated or fused directly or indirectly to a reagent such as a nucleic acid probe and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable

The term “oligonucleotide ligation assay” refers to an assay in which an allele-specific oligonucleotide and a second oligonucleotide are hybridized adjacent to one another on a target nucleic acid and ligated together (either directly or indirectly through intervening nucleotides), and the ligation product is detected directly or indirectly.

The term “genetic bit analysis” or “GBA” refers to a method which involves the incorporation of a nucleotide present at a preselected site onto the terminus of a primer, and their subsequent ligation to a second oligonucleotide. The reaction results in a detectable label attached to the reaction's solid phase or by detection in solution.

The term “assay employing molecular beacons” refers to a relative assay in which hybridization of an allele-specific oligonucleotide to a target nucleic acid results in a level of detectable signal that is higher than the level of detectable signal emitted by the free oligonucleotide.

In another aspect, there is provided method for predicting benefit of a subject having a vascular disease from treatment with a pterin having Formula I, the method comprising the steps of: obtaining a biological sample from the subject, and detecting in the biological sample the presence of a genetic variation in the Actin Alpha 2 gene wherein the genetic variation is the presence of a minor allele of a single nucleotide polymorphism (SNP) selected from the list consisting of: rs112602953, rs112901682, rs121434526, rs121434527, rs121434528, rs387906592, rs397515325, rs397516683, rs397516685, rs 727502878, rs746972765, rs772862676, rs794728019, rs794728021, rs794728025, rs794728029, rs869025352, rs886038852, rs886038978, rs886039303, rs1057521105, rs1060500134, rs1064793016, rs1254836237, rs1554841843 and rs150547139, wherein presence of the substitution is indicative of benefit from treatment with a pterin having Formula I. In some embodiments, the pterin having Formula I

For use in the applications described or suggested above, kits or articles of manufacture are also provided. In some embodiments, a detection kit is provided configured to detect any of the genetic variation identified for selection of patients for pterin Formula I treatment. Such kits may include an organizer being compartmentalized to receive in close confinement one or more container means such as vials, tubes, and the like, each of the container means comprising one of the separate elements to be used in the method e.g., a polynucleotide probe specific for a polynucleotide variation in actin. Where the kit utilizes nucleic acid hybridization to detect the target nucleic acid, the kit may also have containers containing nucleotide(s) for amplification of the target nucleic acid sequence and/or a container comprising a reporter bound to a reporter molecule such as avidin, streptavidin or a biotin-binding protein. Reporter molecules may include but are not limited to enzymatic, fluorescent, or radioisotope label.

Kits will typically include the necessary components described above as well as materials desirable from a commercial and user standpoint, including buffers (e.g., block buffer, wash buffer, substrate buffer, etc.), diluents, filters, needles, syringes, and package inserts with instructions for use. A label may be present on the container to indicate that the composition is used for a specific diagnosis or therapy.

Subsequent to the determination that a subject, or the tissue or cell sample comprises a genetic variation disclosed herein, it is contemplated that a therapeutically effective amount of a pterin derivative Formula I (Ia, Ib or Ic) may be administered to the subject to treat the condition in the subject.

Subsequent to the determination that a subject, or the tissue or cell sample comprises a genetic variation disclosed herein, it is contemplated that a beneficial response may be elicited by administration of a therapeutically effective amount of a pterin Formula I. A “response” of a subject, can be assessed using any endpoint indicating a benefit to the patient, including, without limitation, inhibition, to some extent, of disease progression, including slowing down and complete arrest; reduction in aneurysm size; relief, to some extent, of one or more symptoms associated with the disorder; increase in the length of progression of disease (e.g., increase in aneurysm size) following treatment; and/or decreased mortality at a given point of time following treatment, an increase in functional F-actin, an increase in the ratio between F-actin to G-actin, a decrease in functional G-actin or an increase in contractile force in aortic vascular smooth muscle cells can be detected. In accordance with aspects and embodiments of the disclosure, a therapeutically effective amount of pterin Formula I (Ia or Ib) may be administered. The effective amount may refer to an amount sufficient to cause any one or more of the following in the subject subsequent to treatment: an increase in functional F-actin; an increase in the ratio between F-actin to G-actin; a decrease in functional G-actin; an increase in contractile force in aortic vascular smooth muscle cells; and an improvement in edema.

An “effective amount” or “a therapeutically effective amount” refers to an amount effective, at dosages and for periods of time necessary, to achieve the desired therapeutic or prophylactic result. A “therapeutically effective amount” of a therapeutic agent may vary according to factors such as the disease state, age, sex, and weight of the individual, and the ability of the molecule to elicit a desired response in the individual. A therapeutically effective amount is also one in which any toxic or detrimental effects of the therapeutic agent are outweighed by the therapeutically beneficial effects.

As used herein, administering a therapeutically effective amount refers to administering an amount capable of resulting in some degree of pharmacological effectiveness. In some cases, it may refer to a prophylactically effective amount. A“prophylactically effective amount”may refer to an amount effective, at dosages and for periods of time necessary, to achieve the desired prophylactic result. Typically but not necessarily, since a prophylactic dose is used in subjects prior to or at an earlier stage of disease, the prophylactically affective amount will often be less than the therapeutically effective amount. Pharmacological effectiveness may refer to preventing or delaying development, reducing rate of progression, or treating an aortic aneurysm associated with one or more mutations in an actinA2 gene by administering a therapeutically effective amount of pterin derivative having Formula I (e.g., Ia, Ib or Ic). As used herein, a therapeutically effective amount may refer to preventing or delaying development, reducing rate of progression, or treating an aortic aneurysm.

It will be appreciated that the actual preferred amount of tetrahydropterin (and other active components, where used) to be administered according to the present invention will vary according to the particular tetrahydropterin, the particular composition formulated, and the mode of administration. Many factors that may modify the action of the compound (e.g., body weight, sex, diet, time of administration, route of administration, rate of excretion, condition of the subject, drug combinations, and reaction sensitivities and severities) can be taken into account by those skilled in the art. Administration can be carried out continuously or periodically within the maximum tolerated dose.

Single or multiple dosages may be employed. For example, a therapeutically effective dosage or amount of the pterin derivative Formula I (e.g., Ia, Ib or Ic) may be used alone and may range from about 1 mg/kg to about 100 mg/kg of body weight or more per day. Interspecies scaling of dosages can be performed in a manner known in the art. When in vivo administration of pterin derivative Formula I is employed, normal dosage amounts may vary from about 10 ng/kg to up to 100 mg/kg of mammal body weight or more per day, preferably about 1 μg/kg/day to 10 mg/kg/day, or 1 mg/kg/day to 100 mg/kg/day depending upon the route of administration.

It will also be appreciated that the actual therapeutically effective amount of pterin Formula I may be in a formulation. Formulations of the present invention are prepared by formulating any of the molecules listed in the present disclosure with a pharmaceutically acceptable carrier by conventional procedures into a dosage form suitable for oral, rectal, or parenteral administration. Depending on the dosage form chosen, the carrier used for these pharmaceutical formulations generally includes excipients, binders, disintegrants, antioxidants, buffers, flavors, surfactants, thickeners, lubricants, or any other conventional excipients.

It will also be appreciated that the actual therapeutically effective amount of pterin can be administered in accordance with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Formulations of the present invention may be suitable for oral administration and provided in the form of tablets, sublingual tablets, capsules, powders, granules or fine granules, or suspensions in a non-aqueous liquid such as emulsions, potions or syrups, that contain the prescribed amount of one or more active ingredients.

A desired dosage of the pterin may be administered once a day or may be administered in divided doses of two to four times a day at appropriate intervals.

The pterin may be administered alone without being mixed with other ingredients, or as a formulation containing other active ingredients or excipients. For example, in accordance with aspects and embodiments of the disclosure, the pterin may be formulated in a variety of different ways to promote treatment. The formulation may also include excipients which play a role in formulation, stabilization or contribute to manufacturing of the active agent. The formulation may also include additional agents including any one or more of the following: ascorbic acid (vitamin C), alpha tocopherol (vitamin E), tocopherols (e.g vitamin A), selenium, a beta-carotene, a carotenoid, a flavone, a flavonoid, a folate, a flavanone, an isoflavone, a catechin, an anthocyanidin, and a chalcone. The formulation may include substrates, coenzymes, or cofactors. The proportion of each of said auxiliary active ingredients in formulations of the present invention is not specifically limited.

In some embodiments, the pterin derivative Formula I may be administered or formulated for administration separately, sequentially, or simultaneously with one or more active agent such as an aneurysm inhibitor. In other embodiments, the pterin derivative Formula I may be administered concomitantly in combination with one or more aneurysm inhibitors. In other embodiments, the pterin derivative Formula I may be formulated in combination with one or more aneurysm inhibitors.

The term aneurysm inhibitor refers to any molecule or device which can inhibit development or progression of an aneurysm. Examples include but are not limited to a beta blocker, a calcium channel blocker, an angiotensin II receptor blocker, a statin, and combinations thereof. The beta blocker may be selected from the group consisting of acebutolol, atenolol, betaxolol, bisoprolol, carteolol, labetalol, metoprolol, nadolol, nebivolol, penbutolol, pindolol, propranolol, sotanol, timolol, and combinations thereof. The calcium channel blocker may be selected from the group consisting of amlodipine, beprifil, diltiazem, felodipine, isradipine, nicardipine, nifedipine, nisoldipine, verapamil, and combinations thereof. The angiotensin II receptor blocker may be selected from the group consisting of azilsartan, candesartan, eprosartan, irbesartan, losartan, olmesartan, termisartan, valsartan, and combinations thereof. The statin may be selected from the group consisting of atorvastatin, fluvastatin, lovastatin, pitavastatin, pravastatin, rosuvastatin, simvastatin, and combinations thereof.

While the invention has been disclosed in this patent application by reference to the details of various embodiments of the invention, it is to be clear that the disclosure is intended in an attempt to illustrate rather than to be limiting. It is contemplated that modifications will readily occur to those skilled in the art, within the spirit of the invention and the scope of the appended claims.

### EXAMPLES

### Example 1: Preparation of G-Actin and F-Actin Fraction

Normal cells, cells containing a point mutation in ACTA2 and cells containing a point mutation in ACTA2 and treated with a pterin derivative Formula I are cultured in 100-mm dishes in DCCM media for 24 h. Cells are washed with Phosphate Buffered Saline buffer and lysed with 1 ml of F-actin stabilizing lysis solution containing 50 mM PIPES, pH 6.9, 50 mM NaCl, 5 mM MgCl2, 5 mM egtazic acid, 5% glycerol, 0.1% Triton X-100, 0.1% Tween-20, 0.1% NP-40 (nonionic polyoxyethylene surfactant), 0.1% 2-mercaptoethanol, 1 mM ATP, and protease inhibitor cocktail for 45 min on ice. The cells are scraped and centrifuged at 100,000×g for 1 hour. The supernatant containing the G-actin fraction is collected while the pellet was dissolved in 1 ml of chilled water containing 1 mM cytochalasin D and incubated for 1 hour. The pellet is centrifuged at 13,000 rpm for 30 min, and the supernatant is collected for F-actin fraction. Equal volumes of G-actin and F-actin fraction are subjected for immunoblot analysis using an anti-β-actin antibody [β-actin (C4), Santacruz Antibodies], and imaging is done by Enhanced Chemiluminescence (ECL) using ChemiDoc Imaging Systems, Bio-Rad. The F/G-actin ratio is determined by densitometry of the immunoblots.

The normal cells have a ratio of 1:1 of F to G actin. The ACTA2 point mutation results in a decrease ratio of F to G actin in the mutant cells. However, when treated with sapropterin, the ratio of F to G actin is in between the two prior values.

### Example 2: Animal Mode Testing: Loss of Function of ACTA2 Zebrafish Model

Method: Establishing humanized TAAD mutant zebrafish model carrying loss of function mutant ACTA2 protein (human) and treating with Sapropterin (Kuvan®) in order to rescue the phenotype.

The general method is described in Prendergast A. el. al. 2022 was used to produce site-specific genomic edits in Zebrafish to obtain ACTA2 morpholino knockdown by CRISPR/Cas9 microinjection. Zebrafish were subsequently reintroduced with wildtype (wt) and mutant human mRNAs corresponding to the identified missense mutations. Next, a deleterious substitution in the ACTA2 amino acid sequence (R149C) that is frequently seen in patients with familial ascending thoracic aortic aneurysms was introduced.

Rescue/fail to rescue experiments involving wild type (wt) and mutant mRNAs as well as drug treatment.

Assessment of TAA phenotype: Injected zebrafish were analyzed between 1-6 dpf for potential phenotypes using an Olympus MVX-10 Macro Zoom microscope. Findings searched included: cerebral hemorrhage, aortic hemorrhage, scoliosis/axial curvature, and abnormalities of cardiac and vascular morphology (dorsal aorta (DA) and intersegmental vessels (INV)), cardiac edema or cardiomegaly.

### Rescue TAA Phenotype Using Kuvan (Sapropterin)®

Various concentration of drug treatments were applied in order to rescue the humanized TAAD mutant model fish. Such experiments were be carried out by performing a dose/response curve for 10, 100 and 1000 μM Kuvan® (sapropterin). The effects on the ACTA2 mutant phenotype were examined. The following list of treatments were applied and results are display in the table, moving from left to right columns:
1. Uninjected zebrafish treated with 0.05% DMSO (vehicle)
2. Acta2 morpholino zebrafish+wt ACTA2 RNA treated with 0.05% DMSO (vehicle)
3. Acta2 morpholino zebrafish+R149C ACTA2 RNA treated with 0.05% DMSO (vehicle)
4. Acta2 morpholino zebrafish+R149C ACTA2 RNA treated with 10 μM Kuvan®.
5. Acta2 morpholino zebrafish+R149C ACTA2 RNA treated with 100 μM Kuvan®.
6. Acta2 morpholino zebrafish+R149C ACTA2 RNA treated with 1000 μM Kuvan®.

### Results

The most pronounced defect across all conditions was some degree of cardiac edema or cardiomegaly in zebrafish. Axial curvature was also disrupted. This suggests that knocking out ACTA2 and reintroducing human ACTA2 amino acid sequence (R149C), a known TAA-associated gene produced abnormal vascular phenotypes in larval zebrafish as young as 2 dpf.

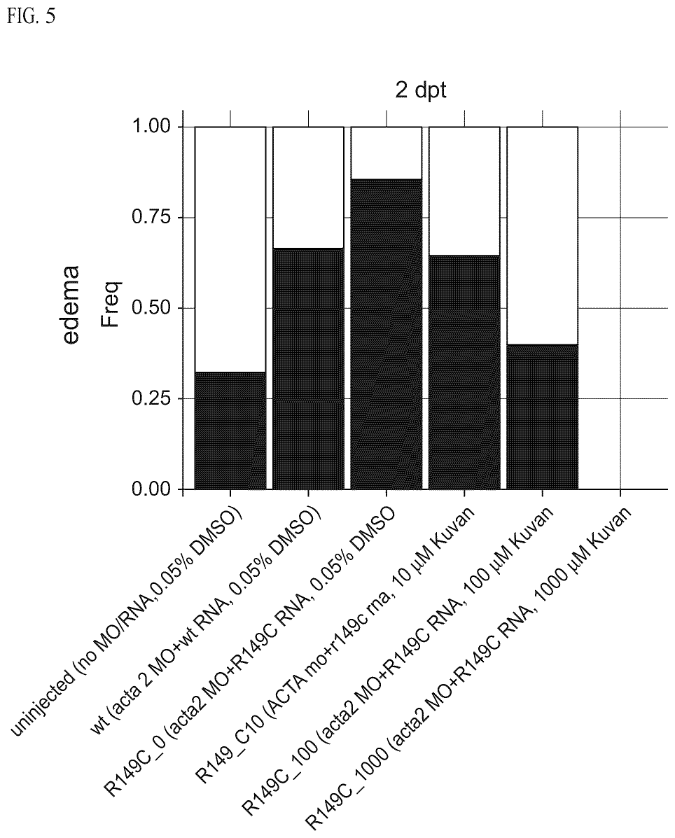

As can be seen in FIG. 5, the incidence of edema is initially higher in the mutant condition than the wt condition and there is indication that it decreases with increasing drug concentration which is suggestive of an overall trend towards treatment by sapropterin.

### Example 3: Patient Study

A 4-year-old female patient was diagnosed with Multisystem smooth muscle dysfunction syndrome (MSMDS), an extremely rare genetic syndrome resulting from a pathogenic heterozygous mutation in the ACTA2 gene (H179R). The mutation in the ACTA2 gene causes uncontrolled proliferation of smooth muscle, which is found in many tissues in the body, and therefore, as a result, damage is caused to many systems in the body. After her birth, she presented with coarctation of the aorta, infarcts in the brain tissue and permanent pupil dilation. Permanent mydriasis (dilation of the pupils), as a result of hypoplasia of the iris sphincter muscles, cardiac involvement (situation after surgery to repair coarctation of the aorta and PDA closure), expansion of the ascending aorta, pulmonary involvement (previously pulmonary hypertension and need for oxygen support), involvement of the digestive system with reduced peristalsis of the intestine and severe constipation, as well as expansion of the urinary bladder. She suffers from exercise intolerance and fatigue during the day, with the need to rest a lot, phenomena well known to patients with this disease.

Neurologically, the patient suffers from a very severe characteristic cerebral vasculopathy with a characteristic picture of “over-alignment” of the arteries, infarcts in the brain tissue and severe white matter disease secondary to small vessel involvement. The cerebral vasculopathy described in the disease additionally includes constrictions and dilatations of the blood vessels to the point of aneurysms in large blood vessels in the anterior and posterior vascular system.

Due to the multi-systemic involvement and the impairment of autoregulation secondary to damage to the muscular media layer of the blood vessels, there is a tendency for blood pressure to drop, which increases the risk of cerebral infarctions and the worsening of pulmonary pressure, therefore general anesthesia and dehydration should be avoided as much as possible.

The expected course of the disease includes the worsening of cerebral vasculopathy over the years with a very high risk of cerebral infarctions and irreversible neurological damage, and life-threatening progressive expansion of the aorta and other arteries to the point of their rupture and death. Without treatment, life expectancy is extremely limited.

From a developmental point of view, the patient has a mild total developmental delay as well as behavioral difficulties, a tendency to repetitiveness with difficulty in transitions and mental-behavioral rigidity. Presents cognition in the norm range and attends a small educational setting.

Since the disease is progressive, the lack of aggravation of the expansions and narrowing in the blood vessels is considered to stop the progression of the disease.

Over the course of three months, the patient received KUVAN® (sapropterin) based on the labeled dosage, known for its safety profile, as compassionate treatment.

### Results

A significant benefit is reported in the patient's well-being, with an improvement in multiple fronts. General examples include: improved physical energy to walk, exercise and play longer without panting and exhaustion, reduced napping, improved digestion (reduced pain and constipation), and reduced pain.

An echocardiogram showed stability in the Z score of the aortic root. Transcranial doppler examination showed stability and even a slight improvement in the flow of the right MCA and left ACA arteries.

TABLE 1
Transcranial doppler

| | T = 0 | T = 4 months |
|---|---|---|
| MCA-rt | 198/109 | 175/86 |
| ACA-rt | 201/106 | 200/100 |
| MCA-lt | 191/101 | 197/88 |
| ACA-lt | 245/142 | 223/127 |

Brain and neck MRI examination after 3 months of treatment demonstrated: stability in terms of white matter disease, stability in terms of the appearance of the MRA compared to the examination a year ago, no new infarcts in the brain tissue, and this after only 3 months of treatment.

Relative stabilization in the ratio between the extracranial internal carotid artery and the intracranial (based on methods in Lauer A, et. al (2021)), in contradistinction to previous tests between years prior where a sharp upward trend was observed in this ratio, indicating a worsening of both the expansion and the narrowing of the internal carotid artery. Abdominal MRA examination shows stability in the diameter of the abdominal aorta, the celiac artery, the SMA, the main renal artery, etc.

TABLE 2
Measurement of Vessel diameter per MRA (Coronal MIP)

| | Three | | | 4 months | 3 months |
|---|---|---|---|---|---|
| | years | | | prior to | after |
| | prior to | Two years | 7 months | initiation | treatment |
| | treat- | prior to | prior to | of | with |
| | ment | treatment | treatment | treatment | Kuvan ® |
| Petrous ICA | 7.2 rt, | 7.4 rt | 7.7 rt, | — | 7.9 rt, |
| | 7.2 lt | 7.5 lt | 8.1 lt | | 8.4 lt |
| Supra clinoid ICA | 3.7 rt, | 2.9 rt, | 3.4 rt, | — | 3.4 rt, |
| | 4.1 lt | 3.9 lt | 3.8 lt | | 4 lt |
| Ratio | R-1.94 | R-2.55 | R- 2.26 | | Rt- 2.32 |
| Petrous/ | L-1.75 | L-1.92 | L-2.13 | | L-2.1 |
| supraclinoid | | | | | |
| ICA | | | | | |
| Ascending aorta | — | — | — | 2.1 mm | 2.1 mm |
| Abdominal aorta | — | — | — | 9.4 mm | 9.8 mm |

The patient did not suffer from any significant side effects.

### REFERENCES

Guo D C, Pannu H, Tran-Fadulu V, Papke C L, Yu R K, Avidan N, Bourgeois S, Estrera A L, Safi H J, Sparks E, Amor D, Ades L, McConnell V, Willoughby C E, Abuelo D, Willing M, Lewis R A, Kim D H, Scherer S, Tung P P, Ahn C, Buja L M, Raman C S, Shete S S, Milewicz D M. Mutations in smooth muscle alpha-actin (ACTA2) lead to thoracic aortic aneurysms and dissections. Nat Genet. 2007 December; 39 (12): 1488-93. doi: 10.1038/ng.2007.6. Epub 2007 Nov. 11. Erratum in: Nat Genet. 2008 February; 40 (2): 255. PMID: 17994018.
Humphrey J D, Schwartz M A, Tellides G, Milewicz D M (2015) Role of mechano-transduction in vascular biology: Focus on thoracic aortic aneurysms and dissections.Circ Res116 (8): 1448-1461.
Lauer A, Speroni S L, Patel J B, Regalado E, Choi M, Smith E, Kalpathy-Kramer J, Caruso P, Milewicz D M, Musolino P L. Cerebrovascular Disease Progression in Patients With ACTA2 Arg179 Pathogenic Variants. Neurology. 2021 Jan. 26; 96 (4): e538-e552. doi: 10.1212/WNL.0000000000011210. Epub 2020 Nov. 16. PMID: 33199432; PMCID: PMC7905785.
Prendergast A, Ziganshin B A, Papanikolaou D, Zafar M A, Nicoli S, Mukherjee S, Elefteriades J A. Phenotyping Zebrafish Mutant Models to Assess Candidate Genes Associated with Aortic Aneurysm. Genes (Basel). 2022 Jan. 10; 13 (1): 123. doi: 10.3390/genes13010123. PMID: 35052463; PMCID: PMC8775119.
Regalado E S, Guo D C, Prakash S, Bensend T A, Flynn K, Estrera A, Safi H, Liang D, Hyland J, Child A, Arno G, Boileau C, Jondeau G, Braverman A, Moran R, Morisaki T, Morisaki H; Montalcino Aortic Consortium; Pyeritz R, Coselli J, LeMaire S, Milewicz D M. Aortic Disease Presentation and Outcome Associated With ACTA2 Mutations. Circ Cardiovasc Genet. 2015 June; 8 (3): 457-64. doi: 10.1161/CIRCGENETICS.114.000943. Epub 2015 Mar. 10. PMID: 25759435; PMCID: PMC4601641.
Wang Z, You Y, Yin Z, Bao Q, Lei S, Yu J, Xie C, Ye F, Xie X. Burden of Aortic Aneurysm and Its Attributable Risk Factors from 1990 to 2019: An Analysis of the Global Burden of Disease Study 2019. Front Cardiovasc Med. 2022 May 31; 9: 901225. doi: 10.3389/fcvm.2022.901225. PMID: 35711350; PMCID: PMC9197430.

1. A method of treating a subject having one or more genetic variations in an Actin alpha 2 (ACTA2) gene, comprising: administering a therapeutically effective amount of sapropterin or a pharmaceutically acceptable salt or/and polymorph thereof to a subject, contingent upon a presence of one or more genetic variations selected from a minor allele of a single nucleotide polymorphism (SNP) selected from a list consisting of: rs112602953, rs112901682, rs121434526, rs121434527, rs121434528, rs397515325, rs397516683, rs397516685, rs727502878, rs746972765, rs772862676, rs794728019, rs794728021, rs794728025, rs794728029, rs869025352, rs886038852, rs886038978, rs886039303, rs1057521105, rs1060500134, rs1064793016, rs 1254836237, rs1554841843 and rs150547139, and restoring a function of the ACTA2 gene.

2. The method of claim 1, wherein the subject is diagnosed with or in a risk category for developing a vascular disease.

3. The method of claim 2, wherein the subject is diagnosed with a vascular disease or in a risk category for developing an aortic aneurysm.

4. The method of claim 1, further comprising obtaining a biological sample from the subject and detecting in the biological sample a presence of the one or more genetic variations being substitutions in an actin alpha (ACTA2) locus.

5. The method of claim 1, wherein the genetic variation is a substitution in an actin alpha (ACTA2) locus selected from: a substitution that results in an amino acid change consisting of a histidine (H) allele of a single nucleotide polymorphism (SNP) of rs121434527; a valine (V) allele of a single nucleotide polymorphism (SNP) of rs397515325; a valine (V) allele of a single nucleotide polymorphism (SNP) of rs397516683; a glutamine (Q) allele of a single nucleotide polymorphism (SNP) of rs397516685; a asparagine (N) allele of a single nucleotide polymorphism (SNP) of rs727502878; a histidine (H) allele of a single nucleotide polymorphism (SNP) of rs746972765; a cysteine (C) allele of a single nucleotide polymorphism (SNP) of rs772862676; a valine (V) allele of a single nucleotide polymorphism (SNP) of rs794728019; a histidine (H) allele of a single nucleotide polymorphism (SNP) of rs794728021; a histidine (H) allele of a single nucleotide polymorphism (SNP) of rs794728025; a leucine (L) allele of a single nucleotide polymorphism (SNP) of rs794728025; a glutamic acid (E) allele of a single nucleotide polymorphism (SNP) of rs794728029; a lysine (K) or threonine (T) allele of a single nucleotide polymorphism (SNP) of rs869025352; a cysteine (C) or serine(S) allele of a single nucleotide polymorphism (SNP) of rs886039303; a glutamine (Q) allele of a single nucleotide polymorphism (SNP) of rs1057521105; a leucine (L) allele of a single nucleotide polymorphism (SNP) of rs1060500134; a serine(S) allele of a single nucleotide polymorphism (SNP) of rs1064793016; a glutamic acid (E) allele of a single nucleotide polymorphism (SNP) of rs1254836237; an arginine (R) allele of a single nucleotide polymorphism (SNP) of rs1554841843; a glutamic acid (E) allele of a single nucleotide polymorphism (SNP) of rs150547139; a proline (P) allele of a single nucleotide polymorphism SNP of rs112602953; a glutamine (Q) allele of a single nucleotide polymorphism SNP of rs112602953; a cysteine (C) allele of a single nucleotide polymorphism SNP of rs121434526; and a cysteine (C) allele of a single nucleotide polymorphism SNP of rs121434528.

6. The method of claim 5, wherein the one or more genetic variation is one or more substitutions in an actin alpha (ACTA2) locus selected from: a substitution that results in an amino acid change consisting of a substitution of arginine (R) for proline (P) at position 118 (R118P) or a proline (P) allele of a single nucleotide polymorphism SNP of rs112602953; a substitution that results in an amino acid change consisting of a substitution of arginine for glutamine at position 118 (R118Q) or a glutamine (Q) allele of a single nucleotide polymorphism SNP of rs112602953; a substitution that results in an amino acid change consisting of a substitution of arginine for cysteine at position 149 (R149C) or a cysteine (C) allele of a single nucleotide polymorphism SNP of rs121434526; a substitution that results in an amino acid change consisting of a substitution of arginine for cysteine at position 258 (R258C) or a cysteine (C) allele of a single nucleotide polymorphism SNP of rs121434528.

7. The method of claim 6, wherein the one or more genetic variations is a presence of a minor allele of a single nucleotide polymorphism (SNP) selected from a list consisting of: rs112602953, rs121434526, or rs121434528.

8. The method of claim 1, wherein the subject is a fetus, a child, or an adult.

9. The method of claim 8, wherein the child is less than one-year old.

10. The method of claim 8, wherein the subject is a fetus and administering is to a pregnant adult to expose a developing fetus.

11. The method of claim 1, wherein the effective amount of Sapropterin is an amount sufficient to cause in the subject, subsequent to treatment, any one or more of the following: an increase in functional F-actin; an increase in a ratio between F (fibrous actin polymerized in the form of a double helix) to G (globular monomeric form) in the subject; degradation or decrease G-actin formation; in the subject; an increase in contractile force in aortic vascular smooth muscle cells; and improvement in edema.

12. The method of claim 2, wherein, the vascular disease is selected from a list consisting of: premature onset coronary artery disease, premature ischemic strokes, Moyamoya disease and aneurysm.

13. The method of claim 12, wherein, the aneurysm is an aortic aneurysm.

14. The method of claim 13, wherein the aortic aneurysm is a descending aortic aneurysm, an ascending aortic aneurysm, and/or an abdominal aortic aneurysm.

15. The method of claim 14, wherein the ascending aortic aneurysm is an ascending thoracic aortic aneurysm.

16. The method of claim 1, wherein administering a therapeutically effective amount of sapropterin is separately, sequentially, or simultaneously with one or more aneurysm inhibitors.

17. The method of claim 16, wherein administering a therapeutically effective amount of sapropterin is in combination with an aneurysm inhibitor.

18. The method of claim 17, wherein the aneurysm inhibitor is selected from a group consisting of a beta blocker, a calcium channel blocker, an angiotensin II receptor blocker, a statin, and combinations thereof.


SEQUENCE LISTING (1 sequence)

SEQ ID NO: 1 - protein, 377 aa, Homo sapiens
MCEEEDSTALVCDNGSGLCKAGFAGDDAPRAVFPSIVGRPRHQGVMVGMGQKDSYVGDEAQSKRGILTLKYPIEHGIITNWDDMEKIWHHSFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF